SEQUENCE LISTING (2 sequences)

SEQ ID NO: 1 - DNA, 33 bp, Mus musculus
gcgtctagaatgtctactgtccacgaaatcctg

SEQ ID NO: 2 - DNA, 32 bp, Mus musculus
cgcggatcctcagtcatccccaccacacaggt

(19) United States
(12) Patent Application Publication
(10) Pub. No.: US 2019/0316124 A1
(43) Pub. Date: Oct. 17, 2019

(54) SEMAPHORIN 3D AND PLEXIN D1 AS THERAPEUTIC TARGETS FOR PANCREATIC CANCER TREATMENT

(71) Applicant: The Johns Hopkins University, Baltimore, MD (US)

(72) Inventor: Lei Zheng, Ellicott City, MD (US)

(21) Appl. No.: 16/320,632

(22) PCT Filed: Jul. 27, 2017
(86) PCT No.: PCT/US17/44263

§ 371 Date: Jan. 25, 2019

Related U.S. Application Data
(60) Provisional application No. 62/367,333, filed on Jul. 27, 2016.

(51) Int. Cl. C12N 15/113; C07K 16/28; C07K 16/18; A61K 45/06; G01N 33/50; G01N 33/574; A61K 39/395; A61P 35/04

(52) U.S. Cl. CPC C12N 15/113; C07K 16/28; C07K 16/18; A61K 45/06; C12N 2310/122; G01N 33/57438; A61K 39/3955; A61P 35/04; C12N 2310/14; G01N 33/5011

(57) ABSTRACT

The invention features compositions and methods for treating and preventing pancreatic cancer.

### CROSS REFERENCE TO RELATED APPLICATIONS

This application claims priority to and the benefit of U.S. Provisional Application No. 62/367,333, filed Jul. 27, 2016, the full disclosure of which is hereby incorporated by reference herein for all purposes.

### STATEMENT OF RIGHTS TO INVENTIONS MADE UNDER FEDERALLY SPONSORED RESEARCH

This work was supported by the National Institutes of Health under grant number CA169702, under grant number CA006973, and under grant number CA062924. The Government has certain rights in this invention.

### FIELD OF THE INVENTION

This invention relates generally to the field of oncology.

### BACKGROUND OF THE INVENTION

Prior to the invention described herein, pancreatic ductal adenocarcinoma (PDA) had a poor prognosis due to late detection and resistance to conventional therapies. As such, there is a pressing need to identify additional treatment options for PDA.

### SUMMARY OF THE INVENTION

The invention is based, at least in part, on the surprising discovery that Semaphorin 3D (Sema3d) autocrine signaling mediates the metastatic role of annexin A2 (AnxA2) in pancreatic cancer. Specifically, PDA metastasis formation is linked to the secretion of Sema3D mediated by AnxA2, which secretion subsequently activates plexin D1 (PlxnD1). Finally, the increase in abundance of Sema3D and PlxnD1 in human PDA metastasis is linked with poorer survival.

Accordingly, provided herein are methods of treating or preventing cancer in a subject comprising administering an agent that reduces the transcription or activity (i.e., inhibits) Sema3D, PlxnD1, and/or AnxA2 in a subject. Specifically, provided are methods of reducing or inhibiting tumor invasion or tumor metastatic progression in a subject comprising identifying a subject having or at risk of developing cancer; and administering to the subject an effective amount of an agent that reduces the transcription or activity of Semaphorin, thereby reducing or inhibiting tumor invasion or tumor metastatic progression in the subject.

Also provided are methods of reducing or inhibiting tumor invasion or tumor metastatic progression in a subject comprising identifying a subject having or at risk of developing cancer; and administering to the subject an effective amount of an agent that reduces the transcription or activity of plexin, thereby reducing or inhibiting tumor invasion or tumor metastatic progression in the subject.

Preferably, the methods described herein inhibit the growth or progression of cancer, e.g., a tumor, in a subject. For example, the methods described herein inhibit the growth of a tumor by at least 1%, e.g., by at least 2/o, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, at least 90% or 100%. In other cases, the methods described herein reduce the size of a tumor by at least 1 mm in diameter, e.g., by at least 2 mm in diameter, by at least 3 mm in diameter, by at least 4 mm in diameter, by at least 5 mm in diameter, by at least 6 mm in diameter, by at least 7 mm in diameter, by at least 8 mm in diameter, by at least 9 mm in diameter, by at least 10 mm in diameter, by at least 11 mm in diameter, by at least 12 mm in diameter, by a least 13 mm in diameter, by at least 14 mm in diameter, by at least 15 mm in diameter, by at least 20 mm in diameter, by at least 25 mm in diameter, by at least 30 mm in diameter, by at least 40 mm in diameter, by at least 50 mm in diameter or more.

An exemplary Semaphorin comprises Sema3D. Suitable agents that reduce the transcription or activity of Sema3D include a small molecule inhibitor, an antibody or a fragment thereof (e.g., an anti-Sema3D monoclonal antibody), or a nucleic acid molecule. For example, the nucleic acid molecule comprises double stranded ribonucleic acid (dsRNA), small hairpin RNA or short hairpin RNA (shRNA), or antisense RNA, or any portion thereof.

A small molecule is a compound that is less than 2000 daltons in mass. Typically, small molecules are less than one kilodalton. The molecular mass of the small molecule is preferably less than 1000 daltons, more preferably less than 600 daltons, e.g., the compound is less than 500 daltons, 400 daltons, 300 daltons, 200 daltons, or 100 daltons.

Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecule inhibitors can be synthetically engineered to consist of a fragment, or small portion, or a longer amino acid chain to fill a binding pocket of an enzyme.

In some cases, the Sema3D inhibitor is administered at a dose of 1 mg/kg/day-1 g/kg/day. For example, the Sema3D inhibitor is administered at a dosage of 0.01-10 mg/kg (e.g., 0.01, 0.05, 0.1, 0.5, 1, 5, or 10 mg/kg) bodyweight. For example, the Sema3D inhibitor is administered in an amount of 0.01-30 mg (e.g., 0.01, 0.05, 0.1, 0.5, 1, 5, 10, 20, or 30 mg) per dose. In another example, the Sema3D inhibitor is administered in the dose range of 0.1 mg/kg to 10 mg/kg of body weight.

Optionally, the method further comprises administering an agent that reduces the transcription or activity of plexin to the subject. An exemplary plexin comprises plexin D1 (PlxnD1). Suitable agents that reduce the transcription or activity of PlxnD1 include a small molecule inhibitor, an antibody or a fragment thereof (e.g., an anti-PlxnD1 monoclonal antibody), or a nucleic acid molecule. For example, the nucleic acid molecule comprises double stranded ribonucleic acid (dsRNA), small hairpin RNA or short hairpin RNA (shRNA), or antisense RNA, or any portion thereof.

In some cases, the PlxnD1 inhibitor is administered at a dose of 1 mg/kg/day-1 g/kg/day. For example, the PlxnD1 inhibitor is administered at a dosage of 0.01-10 mg/kg (e.g., 0.01, 0.05, 0.1, 0.5, 1, 5, or 10 mg/kg) bodyweight. For example, the PlxnD1 inhibitor is administered in an amount of 0.01-30 mg (e.g., 0.01, 0.05, 0.1, 0.5, 1, 5, 10, 20, or 30 mg) per dose. In another example, the PlxnD1 inhibitor is administered in the dose range of 0.1 mg/kg to 10 mg/kg of body weight.

In another aspect, the method further comprises administering an agent that reduces the transcription or activity of AnxA2 to the subject. Suitable agents that reduce the transcription or activity of AnxA2 include a small molecule inhibitor, an antibody or a fragment thereof (e.g., an anti-AnxA2 monoclonal antibody), or a nucleic acid molecule. For example, the nucleic acid molecule comprises double stranded ribonucleic acid (dsRNA), small hairpin RNA or short hairpin RNA (shRNA), or antisense RNA, or any portion thereof.

In some cases, the AnxA2 inhibitor is administered at a dose of 1 mg/kg/day-1 g/kg/day. For example, the AnxA2 inhibitor is administered at a dosage of 0.01-10 mg/kg (e.g., 0.01, 0.05, 0.1, 0.5, 1, 5, or 10 mg/kg) bodyweight. For example, the AnxA2 inhibitor is administered in an amount of 0.01-30 mg (e.g., 0.01, 0.05, 0.1, 0.5, 1, 5, 10, 20, or 30 mg) per dose. In another example, the AnxA2 inhibitor is administered in the dose range of 0.1 mg/kg to 10 mg/kg of body weight.

In some cases, the cancer comprises a gastrointestinal cancer, e.g., pancreatic cancer, e.g., pancreatic ductal adenocarcinoma (PDA).

The subject is preferably a mammal in need of such treatment, e.g., a subject that has been diagnosed with cancer, e.g., pancreatic cancer, or a predisposition thereto, i.e., at risk of developing pancreatic cancer. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Modes of administration include intravenous, systemic, oral, rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy is also provided.

For example, the composition is administered in a form selected from the group consisting of pills, capsules, tablets, granules, powders, salts, crystals, liquids, serums, syrups, suspensions, gels, creams, pastes, films, patches, and vapors.

In some cases, the subject has had the bulk of the tumor resected.

Optionally, the Sema3D inhibitor and the PlxnD1 inhibitor are administered simultaneously. Alternatively, the Sema3D inhibitor and the PlxnD1 inhibitor are administered sequentially. In some cases, the Sema3D inhibitor and the PlxnD1 inhibitor are administered twice per week. In another aspect, the AnxA2 inhibitor is administered simultaneously, or sequentially with the Sema3D inhibitor and/or the PlxnD1 inhibitor.

In some cases, the methods further comprise administering an anti-cancer agent to the subject. For example, the methods include administering chemotherapy, targeted cancer therapy, cancer vaccine therapy, or immunotherapy to the subject. Treatment with immunotherapeutic methods or compositions described herein may be a stand-alone treatment, or may be one component or phase of a combination therapy regime, in which one or more additional therapeutic agents are also used to treat the patient.

In some cases, the methods described herein are used in conjunction with one or more agents or a combination of additional agents, e.g., an anti-cancer agent. Suitable agents include current pharmaceutical and/or surgical therapies for an intended application, such as, for example, cancer. For example, the methods described herein can be used in conjunction with one or more chemotherapeutic or anti-neoplastic agents. In some cases, the additional chemotherapeutic agent is radiotherapy. In some cases, the chemotherapeutic agent is a cell death-inducing agent.

Also provided is a method of screening for a candidate compound which inhibits tumor invasion and/or tumor metastasis comprising contacting a candidate compound with a pancreatic cancer cell; determining a Sema3D secretion level; and identifying the candidate compound as a candidate compound for inhibiting tumor invasion and/or tumor metastasis if the candidate compound inhibits secretion of Sema3D.

Also provided are methods of determining prognosis of a subject with pancreatic cancer. For example, methods of determining whether pancreatic cancer will metastasize in a subject are carried out by obtaining a pancreatic tumor sample from a subject; determining a level of Sema3D, AnxA2, and/or PlxnD1 in the tumor sample; comparing the level of Sema3D, AnxA2, and/or PlxnD1 in the tumor sample to a control level of Sema3D, AnxA2, and/or PlxnD1, wherein an increased level of Sema3D, AnxA2, and/or PlxnD1 in the tumor sample relative to the control level of Sema3D, AnxA2, and/or PlxnD1 indicates the pancreatic cancer will metastasize in the subject. In some cases, the method further comprises administering to the subject an effective amount of an agent that reduces the transcription or activity of Sema3D, AnxA2, and/or PlxnD1, thereby reducing or inhibiting tumor invasion or tumor metastatic progression in said subject.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “antineoplastic agent” is used herein to refer to agents that have the functional property of inhibiting a development or progression of a neoplasm in a human, particularly a malignant (cancerous) lesion, such as a pancreatic cancer. Inhibition of metastasis is frequently a property of antineoplastic agents.

By “agent” is meant any small molecule chemical compound, antibody, nucleic acid molecule, or polypeptide, or fragments thereof.

By “binding to” a molecule is meant having a physicochemical affinity for that molecule.

By “control” or “reference” is meant a standard of comparison. As used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

As used herein, “detecting” and “detection” are understood that an assay performed for identification of a specific analyte in a sample, e.g., an antigen in a sample or the level of an antigen in a sample. The amount of analyte or activity detected in the sample can be none or below the level of detection of the assay or method.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “polynucleotide” or “nucleic acid” as used herein designates mRNA, RNA, cRNA, cDNA or DNA. As used herein, a “nucleic acid encoding a polypeptide” is understood as any possible nucleic acid that upon (transcription and) translation would result in a polypeptide of the desired sequence. The degeneracy of the nucleic acid code is well understood. Further, it is well known that various organisms have preferred codon usage, etc. Determination of a nucleic acid sequence to encode any polypeptide is well within the ability of those of skill in the art.

As used herein, “isolated” or “purified” when used in reference to a polypeptide means that a polypeptide or protein has been removed from its normal physiological environment (e.g., protein isolated from plasma or tissue, optionally bound to another protein) or is synthesized in a non-natural environment (e.g., artificially synthesized in an in vitro translation system or using chemical synthesis). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a synthetic cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

As used herein, “kits” are understood to contain at least one non-standard laboratory reagent for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for t and a isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ), respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a 1-sheet configuration, connected by three hypervariable regions, which form loops connecting, and in some cases forming part of, the 1-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the Vi, and around about 31-35 (H1), 50-65 (H2) and 95-102 (H3) in the Vi when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Also provided are variable domain antigen-binding sequences derived from human antibodies. Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting import hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

An “intact” antibody is one that comprises an antigen-binding site as well as a C_{L} and at least heavy chain constant domains, C_{H}1, C_{H}2 and C_{H}3. The constant domains may be native sequence constant domains (e.g., human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870; Zapata et al., Protein Eng. 8(10): 1057-1062 [1995]); single-chain antibody molecules; and multispecific antibodies formed from antibody fragments.

The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc_{ε}RI.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (Vii), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

As used herein, an antibody that “internalizes” is one that is taken up by (i.e., enters) the cell upon binding to an antigen on a mammalian cell (e.g., a cell surface polypeptide or receptor). The internalizing antibody will of course include antibody fragments, human or chimeric antibody, and antibody conjugates. For certain therapeutic applications, internalization in vivo is contemplated. The number of antibody molecules internalized will be sufficient or adequate to kill a cell or inhibit its growth, especially an infected cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the infected cell.

As used herein, an antibody is said to be “immunospecific,” “specific for” or to “specifically bind” an antigen if it reacts at a detectable level with the antigen, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and in certain embodiments, HuM2e antibody specifically binds to M2e if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of antibodies can be readily determined using conventional techniques, for example, those described by Scatchard et al. (Ann. N.Y. Acad. Sci. USA 51:660 (1949)).

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

The term “antagonist” antibody is used in the broadest sense, and includes an antibody that partially or fully blocks, inhibits, or neutralizes a biological activity of an epitope, polypeptide, or cell that it specifically binds. Methods for identifying antagonist antibodies may comprise contacting a polypeptide or cell specifically bound by a candidate antagonist antibody with the candidate antagonist antibody and measuring a detectable change in one or more biological activities normally associated with the polypeptide or cell.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al., PNAS (USA) 95:652-656 (1998).

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42.degree. C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Obtaining” is understood herein as manufacturing, purchasing, or otherwise coming into possession of.

As used herein, “operably linked” is understood as joined, preferably by a covalent linkage, e.g., joining an amino-terminus of one peptide, e.g., expressing an enzyme, to a carboxy terminus of another peptide, e.g., expressing a signal sequence to target the protein to a specific cellular compartment; joining a promoter sequence with a protein coding sequence, in a manner that the two or more components that are operably linked either retain their original activity, or gain an activity upon joining such that the activity of the operably linked portions can be assayed and have detectable activity, e.g., enzymatic activity, protein expression activity.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body.

Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

In some cases, a composition of the invention is administered orally or systemically. Other modes of administration include rectal, topical, intraocular, buccal, intravaginal, intracisternal, intracerebroventricular, intratracheal, nasal, transdermal, buccal, sublingual within/on implants, or parenteral routes. The term “parenteral” includes subcutaneous, intrathecal, intravenous, intramuscular, intraperitoneal, intracardiac, intracranial, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments). Optionally the peptide further includes one or more modifications such as modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins, Structure and Molecular Properties, 2nd ed., T. E. Creighton, W.H. Freeman and Company, New York (1993); Posttranslational Covalent Modification of Proteins, B. C. Johnson, ed., Academic Press, New York, pgs. 1-12 (1983); Seifter et al., Meth. Enzymol 182:626-646 (1990); Rattan et al., Ann. N.Y. Acad. Sci. 663:48-62 (1992)).

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The term “reduce” or “increase” is meant to alter negatively or positively, respectively, by at least 5%. An alteration may be by 5%, 10%, 25%, 30%, 50%, 75%, or even by 100%.

A “sample” as used herein refers to a biological material that is isolated from its environment (e.g., blood or tissue from an animal, cells, or conditioned media from tissue culture) and is suspected of containing, or known to contain an analyte, such as a protein. A sample can also be a partially purified fraction of a tissue or bodily fluid. A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “subject” as used herein refers to an organism. In certain embodiments, the organism is an animal. In certain embodiments, the subject is a living organism. In certain embodiments, the subject is a cadaver organism. In certain preferred embodiments, the subject is a mammal, including, but not limited to, a human or non-human mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A “subject sample” can be a sample obtained from any subject, typically a blood or serum sample, however the method contemplates the use of any body fluid or tissue from a subject. The sample may be obtained, for example, for diagnosis of a specific individual for the presence or absence of a particular disease or condition.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer is within the ability of those in the art. Subjects suffering from, and suspected of suffering from, a specific disease, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range.

By “reduces” is meant a negative alteration of at least 5%, 10%, 25%, 5⁰%, 75%, or 100%.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Unless specifically stated or obvious from context, as used herein, the term “or” is understood to be inclusive. Unless specifically stated or obvious from context, as used herein, the terms “a”, “an”, and “the” are understood to be singular or plural.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1E shows that ANXA2 is essential for PDA metastasis formation in a transgenic mouse model of PDA. FIG. 1A shows hematoxylin and eosin (H&E) staining of PDA from representative KPC and KPCA^{-/-} mice. FIG. 1B is a chart showing a tabulated summary of histologically confirmed primary PDA and metastases formed in KRAS^{G12D} TP53^{R172H} PDX-1-CRE^{+/+} (KPC) and KRAS^{G12D} TP53^{R172H} PDX-1-CRE^{+/+} ANXA2^{-/-} (KPCA^{-/-}) mice. All mice in both cohorts developed primary PDA. Gross metastases to the liver were observed in 16 of 17 KPC mice with primary pancreatic tumors. However, none of the KPCA^{-/-} mice (0 of 23) developed metastases to the liver (P<0.001, Fisher's exact test). FIG. 1C is a series of phographs showing gross images of a primary pancreatic tumor and liver from a representative 6-month-old KPC mouse. FIG. 1D is a series of photographs showing gross images of a primary pancreatic tumor and liver from a representative 6-month-old KPCA−/− mouse. FIG. 1E is a series of photomicrographs showing H&E staining of PDA from representative KPC and KPCA^{-/-} mice showing invasive metastases in the liver of the KPC mouse but no invasion of the pancreatic tumor into the liver of the KPCA^{-/-} mouse. Scale bars, 200 mm. Images in all panels are representative of at least 17 mice.

FIG. 2A-FIG. 2F shows that the reintroduction of ANXA2 is able to restore the metastatic potential of ANXA2^{-/-} PDA cells. FIG. 2A is a photograph showing Western blotting for AnxA2 in primary pancreatic tumor lines developed from KPC and KPCA^{-/-} mice. Blots are representative of at least three experiments. FIG. 2B is a graph showing Kaplan-Meier analysis of mice that received a hemi-spleen injection of KPC or KPCA^{-/-} cells (n=10 mice per group) (P<0.001, log-rank test). FIG. 2C is a series of photographs showing the detection of gross metastatic lesions in the livers of mice that received splenic injection of KPC or KPCA^{-/-} cells. Images are representative of 10 mice. FIG. 2D is a photograph showing Western blot analysis demonstrating successful knock-in of ANXA2 expression into KPCA^{-/-} cells. β-Actin was used as a loading control. Blots are representative of at least three experiments. FIG. 2E is a graph showing Kaplan-Meier analysis of mice that received a hemi-spleen injection of KPCA^{-/-}+GFP or KPCA^{-/-}+ANXA2 cells (n=11 mice per group) (P<0.001, log-rank test). FIG. 2F is a series of photographs showing formation of liver lesions by KPCA^{-/-}+ANXA2 or KPCA^{-/-}+GFP cells. Scale bars, 20 mm. Images are representative of 11 mice.

FIG. 3A-FIG. 3F show the abundance of Sema3D is differentially regulated in pancreatic tumors from KPCA^{-/-} and KPC mice. FIG. 3A is a plot showing the six genes involved in cell movement (top) and cell morphology and remodeling (bottom) that had the highest fold change difference in gene expression between KPC and KPCA^{-/-} cells (P<0.001, hypergeometric and Fisher's exact tests). FIG. 3B is a graph showing qRT-PCR validation of the microarray data in independent tumor samples obtained from KPC and KPCA^{-/-} mice. Data are mean fold change of KPC versus KPCA^{-/-} normalized to glyceraldehyde-3-phosphate dehydrogenase (GAPDH) amounts from 12 independent tumors per group. FIG. 3C is a photograph of a Western blot analysis of Sema3D and PlxnD1 abundance in KPC, KPCA^{-/-}, and Panc02 pancreatic tumor cell lines. Blots are representative of at least two experiments. FIG. 3D is a series of photomicrographs showing immunofluorescence analysis of Sema3D and PlxnD1 during pancreatic tumor progression. FIG. 3E is a series of photomicrographs showing immunofluorescence staining of Sema3D [fluorescein isothiocyanate (FITC)] in PanINs from KPC and KPCA^{-/-} mice. FIG. 3F is a series of photomicrographs showing immunofluorescence staining of PlxnD1 (FITC) in PanINs from KPC and KPCA^{-/-} mice. Scale bars, 20 mm. Images in FIG. 3D to FIG. 3F are representative of at least 10 mice.

FIG. 4A-FIG. 4E show that AnxA2 controls the secretion of Sema3D from PDA cells, allowing it to interact with its receptor, PlxnD1, in an AnxA2-dependent manner. FIG. 4A is a graph showing Sema3D secretion in KPC and KPCA^{-/-} cells as determined by ELISA (P<0.031 for all, KPC versus KPCA; linear regression analysis), with and without the addition of antibodies against AnxA2. Data are means±SEM from three independent biological replicates. FIG. 4B is a photograph showing coimmunoprecipitation and Western blot analysis of AnxA2 and Sema3D in KPC cells. KPCA^{-/-} cells were used as a control. FIG. 4C is a series of photographs showing coimmunoprecipitation and Western blot analysis of PlxnD1 and Sema3D in KPC cells. Blots in FIG. 4B and FIG. 4C are representative of at least two experiments. FIG. 4D is a series of photographs showing Sema3D-AP binds to NP-1, PlxnD1, and PlxnD1 in the presence of NP-1. FIG. 4E is a series of photographs showing that exogenous Sema3D-AP binds to PlxnD1 on the surface of both KPC and KPCA^{-/-} cells. Sema3E-AP was used as a control. All images were acquired at ×20 magnification. Scale bars, 20 mm. Images in FIG. 4D and FIG. 4E are representative of at least three experiments.

FIG. 5A-FIG. 5F show that Sema3D is involved in invasion and metastasis of PDA and is associated with poor survival. FIG. 5A is a photograph of a Western blot confirming Sema3D knockdown by shRNA. Blots are representative of at least two experiments. FIG. 5B is a graph showing an in vitro invasion assay using Panc02 cells transfected with control or Sema3D shRNA (P=0.036, unpaired t test). Data are means±SEM from three independent biological replicates. FIG. 5C is a series of photographs showing metastasis formation as assessed at day 14 in the livers from mice receiving a hemi-spleen injection of either KPC Ctrl shRNA cells (n=12 mice) or KPC Sema3D shRNA cells (n=13 mice) (P=0.011, Fisher's exact test). Images are representative of at least 12 mice. FIG. 5D Kaplan-Meier analysis of mice receiving a hemi-spleen injection of KPC Ctrl shRNA or KPC Sema3D shRNA cells (n=10 mice per group) (P=0.004, log-rank test). FIG. 5E is a graph showing tumor growth, wherein an ultrasound was used on days 6 and 20 to assess tumor growth after implantation of subcutaneously grown KPC tumors expressing nontargeting (Ctrl) shRNA or Sema3D-targeting shRNA into the pancreas of syngeneic mice. Data are presented as the tumor volumes on day 20 normalized to the tumor volumes on day 6 (n=9 mice per group) (P=0.84, unpaired t test). FIG. 5F is a series of photographs showing representative immunohistochemistry staining of PDAs from a patient with a DFS of >2 years (of 20 patients) and a patient with a DFS<1 year (of 15 patients) (P=0.007, Fisher's exact test). Scale bar, 20 mm. Images are representative of 35 surgically resected human PDAs.

FIG. 6A-FIG. 6C show that overexpression of Sema3D reverses the defect in invasion and metastasis formation in ANXA2-deficient PDA cells. FIG. 6A is a graph showing the invasion potential of Panc02 cells was assessed after siRNA knockdown of ANXA2 expression (P=0.029, Ctrl-AP and Ctrl siRNA versus Ctrl-AP and ANXA2 siRNA; unpaired t test), as well as after siRNA knockdown of ANXA2 and overexpression of Sema3D-AP (P=0.1205, Ctrl-AP and ANXA2 siRNA versus Sema3D-AP and ANXA2 siRNA; unpaired t test). CCK8 was used to quantify the number of invaded cells. Data are means±SEM from four independent biological replicates. FIG. 5B is a series of photographs showing representative H&E staining of livers from mice receiving a hemi-spleen injection of either KPCA^{-/-} +GFP cells or KPCA^{-/-}+Sema3D cells (n=12 mice) (P<0.001, Fisher's exact test). Scale bars, 20 mm. Images are representative of at least 11 mice. FIG. 5C is a schematic showing the proposed interaction between AnxA2, Sema3D, and PlxnD1 in PDA cells. AnxA2 regulates the secretion of Sema3D from PDA cells, allowing it to interact with its receptor, PlxnD1. Invasion and metastasis is induced after Sema3D binding to PlxnD1 on the surface of the PDA cell.

FIG. 7 is a series of photomicrographs showing AnxA2 staining in normal pancreas tissue, PanINs, and PDA from KRAS^{G12D} TP53^{R172H} PDX-1 CRE^{+/+} mice. Representative immunofluorescent staining of AnxA2 (FITC) in normal pancreas tissue, PanINs and primary PDA from KPC mice is shown. Scale bar, 20 μm. Images are representative of at least 10 mice.

FIG. 8A-FIG. 8C is a series of photomicrographs showing histological characterization of PDAs from KPC and KPCA^{-/-} mice. FIG. 8A is a series of photomicrographs showing representative immunohistochemical analysis of Ki67 (proliferation) and TUNEL (apoptosis) in primary PDAs from KPC and KPCA^{-/-} mice. FIG. 8B is a series of photomicrographs showing representative immunohistochemical analysis of CD31 staining (brown signals) in KPC and KPCA^{-/-} primary PDAs is shown. FIG. 8C is a series of photomicrographs showing representative immunofluorescent staining of NG2 (FITC) in KPC and KPCA^{-/-} primary PDAs is shown. Scale bars for all panels, 20 μm. Images in all panels are representative of at least 10 mice.

FIG. 9A-FIG. 9B is a chart and a series of photomicrographs showing KPCA^{-/-} cells have defects in lung metastasis formation in an IVC model of lung metastasis. FIG. 9A is a chart showing that following injection of either KPC or KPCA^{-/-} cells into the IVC of wild-type C57Bl/6 mice, all of the mice receiving KPC cells developed metastases to the lungs at 19 days post-injection. Among them, 8 mice were found with macrometastases in the lungs at necropsy. By contrast, none of mice receiving KPCA^{-/-} cells were found with macrometastases in the lungs (N=11 mice/group) (p<0.001, KPC vs. KPCA^{-/-} macrometastases; Fisher's exact test). Only 2/11 mice receiving KPCA^{-/-} cells were found with micrometastatic foci in the lungs, although the possibility that these foci are aggregates of injected tumor cells cannot be excluded. FIG. 9B is a series of photomicrographs showing representative H&E staining of lung tissue sections from mice injected with KPC or KPCA^{-/-} cells into the IVC. Scale bars, 200 μm. Images are representative of 11 mice.

FIG. 10 is a series of photomicrographs showing that Sema3A localization is unaffected by ANXA2 expression. Representative immunofluorescent staining of Sema3A (FITC) in KPC and KPCA^{-/-} primary PDAs is shown. Scale bars, 20 μm. Images are representative of at least 5 mice.

FIG. 11 is a graph showing that knockdown of ANXA2 in KPC cells results in decreased Sema3D secretion. Sema3D secretion was evaluated by ELISA following knockdown of ANXA2 by siRNA in KPC cells (p<0.001; unpaired t-test). Data are presented as the mean±SEM from 3 independent biological replicates.

FIG. 12A-FIG. 12B is a series of graphs showing that the secretion of Sema3D is mediated by exocytosis and is partially regulated by Tyr23 phosphorylation of AnxA2.

FIG. 12A is a graph wherein Brefeldin A (Golgi Plug) was added to KPC cells for 5 hours to inhibit exocytosis. Sema3D secretion in untreated (NT) and Brefeldin A treated KPC cells was evaluated by ELISA (p<0.001; unpaired t-test). Data are presented as the mean±SEM from 3 independent biological replicates. FIG. 12B is a graph wherein Sema3D secretion was evaluated by ELISA in KPC, KPCA^{-/-}+Y23A-AnxA2 (an AnxA2 mutant that does not localize to the cell surface) and KPCA^{-/-} cells. Data are presented as the mean±SEM from 4 independent biological replicates. Note that the Y23A mutation did not completely abolish the secretion of Sema3D, suggesting that other signaling is also important for Sema3D secretion.

FIG. 13A-FIG. 13B is a series of photomicrographs showing that Sema3D binds to PlxnD1. FIG. 13A is a series of photomicrographs showing representative images of Sema3E-AP binding (purple signal) to COS7 cells transfected with PLXND1 and/or NP-1. FIG. 13B is a series of photomicrographs showing representative images of Sema3D-AP binding (purple signal) to COS7 cells transfected with PLXND1 and/or NP-1. Scale bar for all images, 20 μm. Images are representative of at least 3 experiments.

FIG. 14 is a photograph of a blot showing that Sema3D binds to NP-1 in KPC cells as shown via co-immunoprecipitation and Western blot analysis of NP-1 and Sema3D in KPC cells. Blots are representative of at least 3 experiments.

FIG. 15A-FIG. 15D is a series of line graphs showing that knockdown of Sema3D expression or overexpression of Sema3D does not alter cell proliferation or the rate of tumor growth. FIG. 15A is a line graph wherein proliferation was assessed at 0, 24 and 48 hours in Panc02 cells lentivirally infected with Sema3D-targeting shRNA or scramble shRNA (p=0.55; linear regression). FIG. 15B is a line graph wherein proliferation was assessed at 0, 24 and 48 hours in KPC cells lentivirally infected with Sema3D-targeting shRNA or scramble shRNA. FIG. 15C is a line graph wherein proliferation was assessed at 0, 24 and 48 hours in KPCA−/− cells lentivirally infected with a plasmid containing GFP or full-length Sema3D. Data in panels A-C are presented as the mean±SEM from 3 independent biological replicates. FIG. 15D is a line graph wherein tumor weights were assessed in mice receiving orthotopically implanted KPC tumor cells lentivirally infected with GFP or Sema3D on day 8 following tumor implantation (N=5 mice/group) (p=0.23; unpaired t-test).

FIG. 16 is a series of photomicrographs showing that TGF-β is unable to induce nuclear localization of Snail-1 in Sema3D knockdown PDA cells. Immunofluorescent analysis of Snail-1 was performed in both KPC cells lentivirally infected with Sema3D-targeting shRNA or scramble shRNA with and without prior TGF-β treatment. DAPI was used to stain the nuclei. Scale bar, 20 μm. Images are representative of at least 10 images per condition.

FIG. 17A-FIG. 17B is a series of photographs showing that knockdown of Sema3D expression does not alter primary tumor growth. Mice with KPC tumors orthotopically implanted into their pancreas were examined by ultrasound on day 20 following tumor implantation. FIG. 17A is a photograph showing a representative ultrasound image of the pancreas from a mouse receiving an orthotopic implant of a KPC scramble shRNA tumor. FIG. 17B is a photograph showing a representative ultrasound image of the pancreas from a mouse receiving an orthotopic implant of a KPC Sema3D shRNA tumor. Tumors are indicated by the white arrows. Images are representative of at least 9 mice per group.

FIG. 18 is a graph showing that knockdown of Sema3D does not alter primary tumor growth. Tumor weight was assesses on day 20 following orthotopic implant of KPC tumors with Ctrl shRNA or Sema3D shRNA (N=9 mice/group) (p=0.27; unpaired t-test).

FIG. 19A-FIG. 19D is a series of photographs and charts showing that knockdown of PLXND1 decreases invasion and metastasis of PDA cells. FIG. 19A is a photograph showing PLXND1 knockdown by shRNA was confirmed by Western blot in KPC cells. Beta-actin was used as a loading control. Blots are representative of at least 2 experiments. FIG. 19B is a bar charts wherein the invasion potential of KPC cells was assessed following knockdown of PLXND1 expression using an in vitro invasion assay (p=0.014; unpaired t-test). Data are presented as the mean±SEM from 4 independent biological replicates. FIG. 19C is a chart wherein metastases formation was assessed on day 18 following orthotopic implant of either KPC Ctrl shRNA or KPC PLXND1 shRNA cells (N=10 mice/group) (p=0.011; Fisher's exact test). FIG. 19D is a chart wherein metastases formation was assessed 14 days following a hemi-spleen injection of either KPC Ctrl shRNA or KPC PLXND1 shRNA cells (N=10 mice/group)(p=0.033; Fisher's exact test). Note that fewer metastases were formed by KPC tumors with Ctrl shRNA in this experiment compared to the number observed in FIG. 5, which was likely due to differences in growth kinetics at the time when the tumor cells were implanted into the mice.

FIG. 20A-FIG. 20B is a series of bar charts showing that exogenously overexpressed Sema3D can be secreted from ANXA2-deficient PDA cells. FIG. 20A is a bar chart showing the ratio of secreted AP-tagged Sema3D from KPCA^{-/-} cells to KPC cells following transfection with an AP-tagged Sema3D plasmid. Secreted AP-tagged Sema3D was measured using an alkaline phosphatase assay. FIG. 20B is a bar chart showing the ratio of Sema3D secretion from KPCA^{-/-} cells to KPC cells following infection of lentivirus expressing the full-length Sema3D cDNA. Note that the concentration of endogenously secreted Sema3D from KPCA^{-/-} cells is approximately 1% of that of KPC cells (FIG. 4A); by contrast, the amount of exogenously overexpressed, secreted Sema3D from KPCA^{-/-} cells is increased to about 30-40% of KPC cells. Data are presented as the mean from 3 independent biological replicates.

FIG. 21 is a bar chart showing that exogenous addition of Sema3D-AP to the culture medium partially restores the ANXA2 siRNA-suppressed invasion capacity of Panc02 cells in a PlxnD1-dependent manner. Sema3D-AP secreted by COS7 cells was added to Panc02 cells following siRNA knockdown of ANXA2 (p=0.02, AnxA2 siRNA/Control-AP vs. AnxA2 siRNA/Sema3D-AP; unpaired t-test) or both ANXA2 and PLXND1 (AnxA2+PlxnD1 siRNA). Data are presented as the mean±SEM from 3 independent biological replicates.

FIG. 22 is a photograph wherein liver metastases can be visualized by ultrasound. A representative ultrasound image of a liver following a hemi-spleen injection of KPCA^{-/-} cells expressing full-length Sema3D cDNA is shown. Tumor is indicated by the white arrows. Image is representative of 12 mice.

FIG. 23 is a photograph wherein liver micrometastases are detectable by H&E analysis of liver sections. A representative H&E image of a liver micrometastases (arrow) is shown following a hemi-spleen injection of KPC tumor cells. Scale bar, 20 μm. Image is representative of at least 10 mice.

### DETAILED DESCRIPTION

The invention is based, at least in part, on the surprising discovery that Sema3D autocrine signaling mediates the metastatic role of AnxA2 in pancreatic cancer. As described in detail below, antibodies against a metastasis-associated protein, namely AnxA2, are identified in PDA patients. These patients demonstrated prolonged and recurrence-free survival after resection of the primary tumour. As described herein, metastases were suppressed in a tumor model due to an antibody-mediated blockade of AnxA2. Human PDA genome studies have uncovered genetic alterations of molecular pathways that may regulate the process of metastasis.

As described in detail below, genes encoding Semaphorins and their receptors were found to be among the pathways that were most frequently altered at genetic level in PDA. Semaphorins are molecules that guide nerve fibres, so called axons. Besides Semaphorins, plexins are other axon guidance molecules. Plexins play a role in the development and progression of other cancer types. For instance, PlxnD1 plexin abundance is associated with high-grade primary and metastatic melanomas, a very malignant form of skin cancer. Sema3D and PlxnD1 promote metastasis in various types of cancer.

Described herein are the mechanisms through which Sema3D and PlxnD1 signalling functions are responsible for development and metastasis formation in PDA. As described in detail below, to evaluate the mechanisms by which AnxA2 influences the development of PDA and the occurrence of metastases, a mouse model with two types of mice was designed. The KPC mice developed PDA without AnxA2 blockade and KPCA^{-/-} mice also developed PDA, but with AnxA2 blockade. In 16 of the 17 KPC mice, metastatic lesions were observed in the liver, lungs, or abdominal cavity. However, no observable gross metastatic lesions were seen in the 23 KPCA^{-/-} mice. So, despite the presence of PDA tumors that grow relatively close to the liver in both mice, only mice with PDA tumors that expressed AnxA2 were able to invade and metastasise into the liver.

As described in detail below, these same mice where then used to investigate the downstream pathways that mediate the function of AnxA2 in PDA metastasis formation. Genes of particular interest were Sema3D and PlxnD1 because they belong to gene families that are frequently amplified and mutated in PDA. It was identified that in KPCA^{-/-} mice, the protein abundance of Sema3D was decreased compared to KPC mice. However, the protein abundance of PlxnD1 was similar in both mice. As described herein, in the absence of AnxA2, the secretion of Sema3D was diminished. These data presented herein support a role for AnxA2 in regulating the secretion of Sema3D from PDA cells. To understand how AnxA2 mediates the secretion of Sema3D, the protein-protein interaction between Sema3D and AnxA2 was examined in PDA cells. In KPCA^{-/-} mice lacking AnxA2, the secretion of Sema3D was diminished so no Sema3D would bind to PlxnD1 on the surface of the cell. The data suggest that AnxA2 is required for Sema3D and PlxnD1 to form a complex, likely through controlling the secretion of Sema3D from PDA cells. This then would facilitate the subsequent interaction between Sema3D and PlxnD1 in the surface of the tumor cell.

As described in detail below, because both Sema3D and PlxnD1 are involved in cell motility, it was also examined whether Sema3D is involved in PDA invasion and formation of metastasis. It was identified that Sema3D has a role in controlling PDA invasion and metastasis formation. In conclusion, these results suggest that Sema3D and PlxnD1 represent an AnxA2-downstream pathway that mediates the role of AnxA2 in PDA invasion and the formation of metastasis.

As described in detail below, to further establish the role of Sema3D in PDA metastasis formation, Sema3D immunohistochemistry was performed on human PDA tissue specimens. Tissue specimens of resected PDA presenting abundant Sema3D were observed in 15 of 20 patients (75%). These patients had a disease-free survival of less than 1 year.

Only 4 of 15 patients (26.7%) with abundant Sema3D in their tissue specimens of resected PDA had a disease-free survival of more than 2 years. These data also suggest that Sema3D abundance in PDA is significantly associated with early recurrence after surgical resection. In all PDAs examined, Sema3D abundance was positively correlated with PlxnD1 abundance, suggesting that Sema3D and PlxnD1 may be co-regulated. Furthermore, 14 of 22 patients (63.6%) with widely metastatic disease demonstrated abundant Sema3D in their primary PDA tumour. Also, 17 of 22 patients (77.3%) demonstrated abundant Sema3D in their metastatic tumour. These results suggest that Sema3D is preferentially enriched in metastatic or primary PDA tumors from patients that have a poor prognosis or patients who died with widely metastatic disease.

As described herein, one mechanism of PDA metastasis formation is linked to the secretion of Sema3D mediated by AnxA2. The secretion of Sema3D subsequently activates PlxnD1. The immunohistochemistry studies, linking the increase in abundance of Sema3D and PlxnD1 in human PDA metastasis with poorer survival, provide evidence suggesting that they are important for human PDA metastasis development. As described in detail below, AnxA2 regulates the function of Sema3D by controlling its secretion. Sema3D present outside the cell will bind PlxnD1 on the surface of the PDA tumor cells. In summary, Sema3D, Plexin D1, and Annexin A2 are therapeutic targets for pancreatic cancer.

Biologic materials generated through this study include the KPC transgenic mouse strain (backcrossed for 9 generations between the original KPC mice and C57BL6 mice; the KPC mice originally developed by the David Tuveson group and deposited at the Jackson Laboratory); the KPCA transgenic mouse stain (new strain, crossed between KPC and annexin A2 knock out mice; annexin A2 knock out mice came from Cornell University through an MTA); tumor cell line derived from KPCA mice (KPCA cells); tumor cell line derived from KPC mice (a.k.a. KPC cells); cancer associated fibroblasts derived from KPC and KPCA mice; lentiviral and plasmid constructs expressing annexin A2, Sema3d and plexin D1.

Pancreatic ductal adenocarcinoma (PDA), a devastating malignant disease with a 5-year survival of less than 5%, is highly metastatic and resistant to most conventional chemotherapeutics (1). Surgical resection remains the primary treatment for PDA, but only 20% of patients present with locally resectable disease at the time of diagnosis, and most patients develop drug resistant metastatic disease after surgical resection (2).

The antibodies against a metastasis-associated protein, annexin A2 (AnxA2), were recently identified in the sera of patients who were treated in a phase 2 study with an allogeneic, granulocyte-macrophage colony-stimulating factor (GM-CSF)-secreting tumor vaccine and who demonstrated prolonged recurrence-free survival after surgical resection of primary PDAs (3). In another study, the phosphorylation of Tyr23 in AnxA2 promoted metastases of PDA cells, whereas short hairpin RNA (shRNA)-mediated knockdown or antibody-mediated blockade of AnxA2 suppressed metastases in two murine transplantable tumor models (4).

Human PDA genome studies uncovered genetically altered molecular pathways that may regulate the metastatic process (5, 6). Although originally identified and characterized as axon guidance genes, genes encoding Semaphorins and their cognate receptors (complexes composed of plexins and neuropilins) were found to be among the cellular pathways that are most frequently altered at the genetic level in PDA (5). The genes encoding axon guidance molecules, including class 3 Semaphorins and plexins, were amplified in 18% of PDAs, and an additional 3% of PDAs had mutations in these genes. Previously, amplification of the gene encoding Sema3A and the gene encoding a member of its receptor plexin A1 (PlxnA1) correlated with poor survival in PDA patients (7). Biankin et al. (5) found that the abundance of multiple Semaphorins increased progressively during pancreatic tumorigenesis in a mouse model of PDA, further suggesting that dysregulation of these molecules contributes to PDA progression.

In addition to their known correlation with PDA survival, plexins also play a role in the development and progression of other cancer types. Specifically, PlxnD1 abundance is associated with high-grade primary and metastatic melanomas (8) as well as poorly differentiated cervical carcinoma tissues (9). By serving as a cellular receptor and signaling transducer for the class 3 Semaphorin Sema3E, PlxnD1 promotes cancer cell invasiveness in multiple human tumor types and metastatic spreading in mouse models (10). In addition, Sema3E-PlxnD1 signaling suppresses apoptosis in metastatic breast cancer cells (11). These outcomes of increased PlxnD1 signaling are similar to those implicated for AnxA2 in PDA development (4). Like these axon guidance pathways, Semaphorin 3D (Sema3D) via PlxnD1 has been implicated in angiogenesis, invasion, cancer cell growth, and survival (12). Additionally, Sema3D and PlxnD1 have been shown to promote metastasis in various types of cancer and regulate the epithelial to mesenchymal transition (EMT) (13-15). Here, the mechanisms through which Sema3D-PlxnD1 signaling functions in PDA development and metastasis formation was investigated.

### Pancreatic Ductal Adenocarcinoma

Pancreatic ductal adenocarcinoma (PDA) is the fourth leading cause of cancer related deaths in the United States (Siegel et al., 2014 Cancer Statistics, 64(1):9-29). Over 80% of those diagnosed with PDA are ineligible for curative resection and five-year survival is less than 5% (Moon et al., 2006 Pancreas, 32(1):37-43; Ma et al., 2013 Journal of the National Cancer Institute, 105(22):1694-700).

The symptoms at diagnosis vary according to the location of the cancer in the pancreas, which anatomists divide (from left to right on most diagrams) into the thick head, the neck, and the tapering body, ending in the tail. Regardless of a tumor's location, the most common symptom is unexplained weight loss, which may be considerable. A large minority (between 35% and 47%) of people diagnosed with the disease will have had nausea, vomiting or a feeling of weakness. Tumors in the head of the pancreas typically also cause jaundice, pain, loss of appetite, dark urine, and light-colored stools. Tumors in the body and tail typically also cause pain. People sometimes have recent onset of atypical type 2 diabetes that is difficult to control, a history of recent but unexplained blood vessel inflammation caused by blood clots (thrombophlebitis) known as Trousseau sign, or a previous attack of pancreatitis. A physician may suspect pancreatic cancer when the onset of diabetes in someone over 50-years-old is accompanied by typical symptoms such as unexplained weight loss, persistent abdominal or back pain, indigestion, vomiting, or fatty feces. Jaundice accompanied by a painlessly swollen gallbladder (known as Courvoisier's sign) may also raise suspicion, and can help differentiate pancreatic cancer from gallstones.

Medical imaging techniques, such as computed tomography (CT scan) and endoscopic ultrasound (EUS) are used both to confirm the diagnosis and to help decide whether the tumor can be surgically removed. Magnetic resonance imaging and positron emission tomography may also be used, and magnetic resonance cholangiopancreatography may be useful in some cases. Abdominal ultrasound is less sensitive and will miss small tumors, but can identify cancers that have spread to the liver and build-up of fluid in the peritoneal cavity (ascites). A biopsy by fine needle aspiration, often guided by endoscopic ultrasound, may be used where there is uncertainty over the diagnosis. Liver function tests can show a combination of results indicative of bile duct obstruction (raised conjugated bilirubin, γ-glutamyl transpeptidase and alkaline phosphatase levels).

The most common form of pancreatic cancer (adenocarcinoma) is typically characterized by moderately to poorly differentiated glandular structures on microscopic examination. There is typically considerable desmoplasia or formation of a dense fibrous stroma or structural tissue consisting of a range of cell types (including myofibroblasts, macrophages, lymphocytes and mast cells) and deposited material (such as type I collagen and hyaluronic acid). This creates a tumor microenvironment that is short of blood vessels (hypovascular) and oxygen (tumor hypoxia).

Current treatment modalities, including surgical resection, chemotherapy, and radiation have failed to significantly improve PDA survival in the last 30 years. Thus, prior to the invention described herein, new treatment modalities were desperately needed (Ma et al., 2013 Journal of the National Cancer Institute, 105(22):1694-700).

### Annexin 2

ANXA2 is a involved in tumor metastases and is a potential antigenic target for cancer immunotherapy (Foley, K. et al. PLoS ONE 2011; 6(4): e19390.; Jaffee, E M. OncoImmunology 2012; 1(1):112-114.) The ANXA2 antigen is described in Zheng L et al., 2011 PLoS ONE 6(4): e19390 and Zheng L and Jaffee E M 2012 OncoImmunology, 1(1): 112-114, each of which is incorporated herein by reference.

Annexin 2 (ANXA2) refers in particular to, e.g., Homo sapiens Annexin 2. Human Annexin 2 mRNA is set forth in GenBank Accession No. BC093056 (BC093056.1), incorporated by reference herein in its entirety. Human ANXA2 protein is provided in Genbank Accession No. AAH93056 (AAH93056.1), incorporated herein by reference it its entirety.

### Semaphorin

Semaphorins are a class of secreted and membrane proteins that were originally identified as axonal growth cone guidance molecules. They primarily act as short-range inhibitory signals and signal through multimeric receptor complexes. Semaphorins are usually cues to deflect axons from inappropriate regions, especially important in neural system development. The Semaphorins are grouped into eight major classes based on structure and phylogenetic tree analyses. Classes 1 and 2 are found in invertebrates only, while classes 3, 4, 6, and 7 are found in vertebrates only. Class 5 is found in both vertebrates and invertebrates, and class V is specific to viruses. Classes 1 and 6 are considered to be homologues of each other; they are each membrane bound in invertebrates and vertebrates, respectively. The same applies to classes 2 and 3; they are both secreted proteins specific to their respective taxa. Each class of Semaphorin has many subgroups of different molecules that share similar characteristics.

Homo sapiens Semaporin 3D (Sema3D) mRNA is set forth in GenBank Accession No. NM_152754 (NM_152754.2), incorporated herein by reference in its entirety. Human Sema3D protein is provided in Genbank Accession No. NP_689967 (NP_689967.2), incorporated by reference herein in its entirety.

### Plexin

The major class of proteins that act as Semaphorin receptors are called plexins, with neuropilins as their co-receptors in many cases. Plexins have established roles in regulating Rho-family GTPases. Recent work shows that plexins can also influence R-Ras, which, in turn, can regulate integrins. Such regulation is probably a common feature of Semaphorin signalling and contributes substantially to understanding of Semaphorin biology.

Homo sapiens plexin D1 (PlxnD1) mRNA is set forth in GenBank Accession No. NM_015103 (NM_015103.2), incorporated herein by reference in its entirety. Human PlxnD1 protein is provided in Genbank Accession No. NP_055918 (NP_055918.2), incorporated herein by reference in its entirety.

As described herein, most patients with pancreatic ductal adenocarcinoma (PDA) present with metastatic disease at the time of diagnosis or will recur with metastases after surgical treatment. Semaphorin-plexin signaling mediates the migration of neuronal axons during development and of blood vessels during angiogenesis. As described in detail below, the expression of the gene encoding Semaphorin 3D (Sema3D) is increased in PDA tumors, and the presence of antibodies against the pleiotropic protein annexin A2 (AnxA2) in the sera of some patients after surgical resection of PDA is associated with longer recurrence-free survival. As described herein, by knocking out AnxA2 in a transgenic mouse model of PDA (KPC) that recapitulates the progression of human PDA from premalignancy to metastatic disease, it was identified that AnxA2 promoted metastases in vivo. The expression of AnxA2 promoted the secretion of Sema3D from PDA cells, which coimmunoprecipitated with the co-receptor plexin D1 (PlxnD1) on PDA cells. As described herein, mouse PDA cells in which Sema3D was knocked down or ANXA2-null PDA cells exhibited decreased invasive and metastatic potential in culture and in mice. However, restoring Sema3D in AnxA2-null cells did not entirely rescue metastatic behavior in culture and in vivo, suggesting that AnxA2 mediates additional prometastatic mechanisms. Patients with primary PDA tumors that have abundant Sema3D have widely metastatic disease and decreased survival compared to patients with tumors that have relatively low Sema3D abundance. Thus, as described in detail below, AnxA2 and Sema3D are new therapeutic targets and prognostic markers of metastatic PDA.

Prior to the invention described herein, PDA was rarely controlled or cured by therapeutic interventions because of a minimal understanding of the biologic processes that control its development, invasion, and metastasis formation. The results provided herein elucidate one mechanism of PDA metastasis formation that is mediated by AnxA2-dependent Sema3D secretion and subsequent autocrine activation of PlxnD1. A previous study demonstrated that AnxA2 is involved in PDA metastasis formation in two murine tumor transplant models (4). Here, transgenic mice, which are genetically programmed to spontaneously develop PDA tumors in the same manner as human PDA, crossed with mice that have the ANXA2 gene knocked out were used to enhance the evidence supporting a role for AnxA2 in PDA metastasis formation. As described in detail below, although knockout of ANXA2 prevented metastases to liver and lungs, it did not alter the development of premalignant lesions and primary PDAs in this model, suggesting that the role of AnxA2 in metastasis may be mediated through a metastasis-specific pathway. Metastasis-specific pathways have been reported in breast cancer, (28, 29) but have not yet been defined by the genetic engineered mouse model of PDA. These observations could be due to secondary biological changes; however, these results are consistent with the previously reported studies in which RNA interference of ANXA2 produced a similar effect (4).

As described herein, Sema3D and PlxnD1 were prioritized for further studies because these genes were identified as frequently altered at the genetic level in human PDAs (5). The immunohistochemistry studies correlating the increase in abundance of Sema3D and PlxnD1 in human PDA metastases with poorer survival provide evidence suggesting that they likely are important for human PDA metastasis development. However, they are not the only downstream mediators. Overexpression of Sema3D did not completely reverse the defects in PDA invasion and metastasis formation under ANXA2 knockout or knockdown conditions, although it is also possible that overexpression of Sema3D could not fully restore the abundance and kinetics of Sema3D secretion.

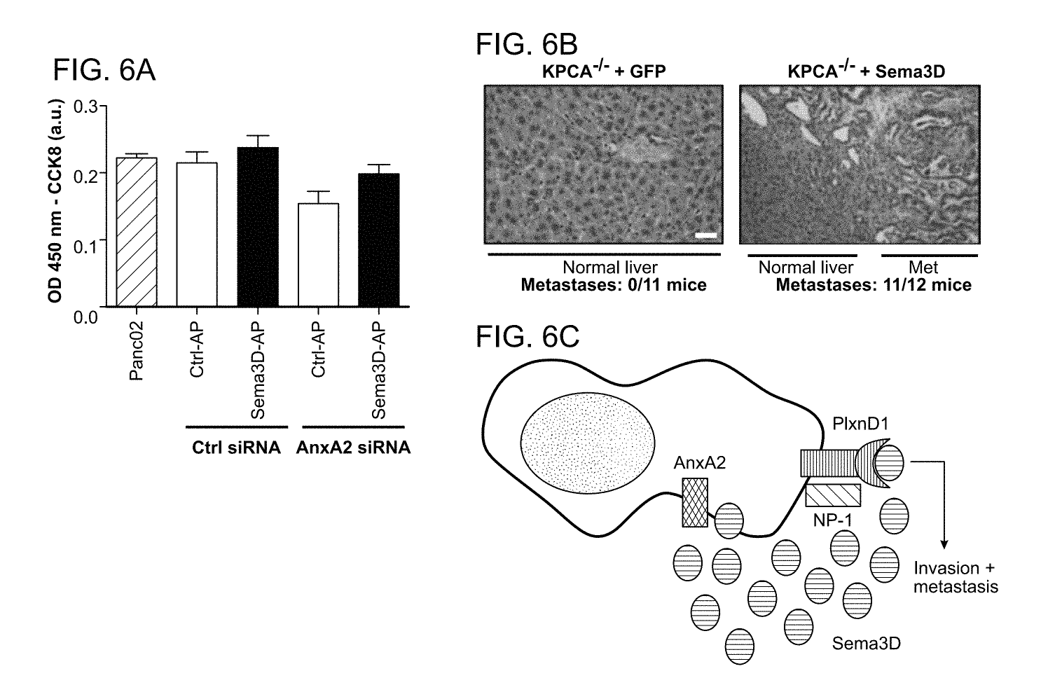

The results presented herein show that AnxA2 regulates the autocrine function of Sema3D by controlling its secretion, which makes Sema3D available in the extracellular space, where it binds PlxnD1 on the surface of PDA tumor cells (FIG. 6C). However, overexpression of Sema3D was able to largely restore the function of Sema3D in the absence of AnxA2. Thus, experiments are performed to delineate the mechanism and extent to which AnxA2 controls Sema3D secretion. Possibilities include genetic regulation of the gene and altered transport of the molecule within the tumor cell. Class 3 Semaphorins colocalize with secretory vesicle proteins, such as Synaptobrevin (30), and AnxA2 has been implicated in vesicle trafficking and exocytosis (31). Thus, given these roles and the observation that Sema3D secretion is decreased after inhibition of exocytosis, it is possible that AnxA2 regulates the packaging of Sema3D into vesicles.

Additional studies clarify how the interaction of Sema3D with PlxnD1 functionally promotes tumor metastases. It is possible that Sema3D's autocrine function regulates PDA cell motility, considering the known functions of Semaphorins and plexins in axon repulsion (32) as well as previous report showing that AnxA2 regulates cell motility and EMT in both human and mouse PDA cells (4). Additionally, it is possible that Sema3D may also act through a paracrine pathway because PlxnD1, its putative co-receptor, is also found on lymphovascular vessels and nerves (25, 33). A paracrine mechanism is also studied because lymphovascular invasion and perineural invasion are two poor prognostic factors (34, 35) and are also proposed to be routes for cancer cells to metastasize along blood vessels, lymphatic vessels, and nerves (34, 35). Additionally, it is explored whether the downstream signaling pathways that mediate the role of PlxnD1 in axon repulsion also mediate the role of AnxA2-Sema3D-PlxnD1 signaling in PDA invasion and metastasis. Previously, it was shown that the Sema3E-PlxnD1 interaction activates a signaling cascade downstream to the phosphorylation of the epidermal growth factor receptor family member ErbB2, specifically through activation of mitogen-activated protein kinase (MAPK) and phospholipase C-g, which subsequently drives invasion and metastasis (10). Thus, it is explored whether the Sema3D-PlxnD1 interaction also activates this signaling cascade during the PDA metastatic process. In summary, this study has revealed a mechanistic role of axon guidance genes in PDA metastasis. Additional studies identify the exact processes by which Sema3D and PlxnD1 induce invasion of PDA cells from the primary tumor site into the surrounding blood vessels, nerves, and lymphatic vessels. This study provides a strong rationale for the development of new therapies targeting AnxA2 and Sema3D as an adjuvant treatment for PDA after local resection.

### Pharmaceutical Therapeutics

The invention provides pharmaceutical compositions for use as a therapeutic. In one aspect, the composition is administered systemically, for example, formulated in a pharmaceutically-acceptable buffer such as physiological saline. Preferable routes of administration include, for example, instillation into the bladder, subcutaneous, intravenous, intraperitoneal, intramuscular, or intradermal injections that provide continuous, sustained levels of the composition in the patient. Treatment of human patients or other animals is carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia or infection, although in certain instances lower amounts will be needed because of the increased specificity of the compound. A compound is administered at a dosage that enhances an immune response of a subject, or that reduces the proliferation, survival, or invasiveness of a neoplastic cell as determined by a method known to one skilled in the art.

### Formulation of Pharmaceutical Compositions

The administration of compositions for the treatment of cancer may be by any suitable means that results in a concentration of the therapeutic that, combined with other components, is effective in ameliorating, reducing, or stabilizing cancer. The composition may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intravesicularly or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice or nonhuman primates, as a skilled artisan recognizes it is routine in the art to modify the dosage for humans compared to animal models. In certain embodiments it is envisioned that the dosage may vary from between about 0.1 μg compound/kg body weight to about 5000 μg compound/kg body weight; or from about 1 μg/kg body weight to about 4000 μg/kg body weight or from about 10 μg/kg body weight to about 3000 μg/kg body weight. In other embodiments this dose may be about 0.1, 0.3, 0.5, 1, 3, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 μg/kg body weight. In other embodiments, it is envisaged that doses may be in the range of about 0.5 μg compound/kg body weight to about 20 μg compound/kg body weight. In other embodiments the doses may be about 0.5, 1, 3, 6, 10, or 20 mg/kg body weight. Of course, this dosage amount may be adjusted upward or downward, as is routinely done in such treatment protocols, depending on the results of the initial clinical trials and the needs of a particular patient.

Pharmaceutical compositions are formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Kits

The invention provides kits for the treatment or prevention of a PDA. In one embodiment, the kit includes a therapeutic or prophylactic composition containing an effective amount of an agent described herein. In some embodiments, the kit comprises a sterile container that contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired an agent of the invention is provided together with instructions for administering the agent to a subject having or at risk of developing a cancer. The instructions will generally include information about the use of the composition for the treatment or prevention of a cancer. In other embodiments, the instructions include at least one of the following: description of the therapeutic agent; dosage schedule and administration for treatment or prevention of a cancer or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1: Materials and Methods

### Human PDA Specimens

Archived PDA specimens were obtained from consecutive patients who underwent pancreaticoduodenectomy between 1998 and 2004 at Johns Hopkins Hospital (JHH) and received adjuvant chemoradiation therapy as previously described (36). Only patients who were primarily followed at JHH, with a DFS of >2 or <1 years, and whose archived paraffin-embedded tissue blocks were in good condition were included. In addition, a tissue microarray made from PDA specimens that were obtained from a JHMI IRB-approved rapid autopsy protocol was also included as previously described (37, 38).

### Mouse Models of PDA

All mice were monitored twice a day. A genetically engineered mouse model of PDA, designated KPC mice, was previously established through a knock-in of pancreatic-specific, conditional alleles of the KRAS^{G12D} and TP53^{R172H} mutations on a mixed 129/SvJae/C57Bl/6 background. These mice, when crossed with PDX-1-CRE^{+/+} mice, develop PanIN lesions that progress stepwise, similar to human disease, into PDA (16). The KPC mice were successfully backcrossed onto a C57Bl/6 background for nine generations. In addition to the KPC mice, ANXA2 homozygous knockout mice (ANXA2−/−) on a C57Bl/6 background were also obtained (17) and crossed with the KPC mice to generate KRAS^{G12D}TP53^{R172H} PDX-1-CRE^{+/+} ANXA2^{-/-} (KPCA^{-/-}) mice.

The mouse hemi-spleen liver metastasis model has been previously described (19, 39). In short, the spleens of anesthetized female C57Bl/6 mice of ages 8 to 10 weeks were divided into two halves, and the halves were clipped. In total, 2×10⁶ PDA cells were injected into the splenic

vessels (splenic artery and veins) through one hemi-spleen followed by a flush of phosphate-buffered saline (PBS) buffer. After the injection, the splenic vessels draining the injected hemi-spleen were clipped, and the hemi-spleen was removed. The abdominal wall was sutured, and the skin was adapted using wound clips. All mice were followed twice daily for survival.

The mouse pancreatic orthotopic model was described previously (4). In brief, 2×10⁶ PDA cells were subcutaneously injected into the flanks of syngeneic female C57Bl/6 mice. After 1 to 2 weeks, the subcutaneous tumors were harvested and cut into ˜1-mm³ pieces. New syngeneic female C57BV/6 mice, ages 8 to 10 weeks, were anesthetized. The abdomen was opened via a left subcostal incision. A small pocket was prepared inside the pancreas using microscissors, into which one piece of the subcutaneous tumor was implanted. The incision in the pancreas was closed with a suture. The abdominal wall was sutured, and the skin was adapted using wound clips. Tumor size and metastasis formation were monitored at the indicated time points using small-animal ultrasound (Vevo770, VisualSonics).

### Inferior Vena Cava Model of Lung Metastases

The inferior vena cava (IVC) of anesthetized female C57Bl/6 mice of ages 8 to 10 weeks was exposed by making a midline incision into the peritoneum and moving the small and large intestines to one side. In total, 5×10⁵ KPC or KPCA^{-/-} cells were injected into the IVC at a position above the superior mesenteric vein. A sterile cotton swab was used to apply pressure for 2 to 3 min immediately after the injection to allow the blood to clot. The abdominal wall was sutured, and the skin was adapted using wound clips. All mice were followed twice daily. The mice were sacrificed 19 days after the IVC injection, and the lungs were harvested for histological analysis of metastasis formation. This model produces lung metastases more consistently than tail vein injection.

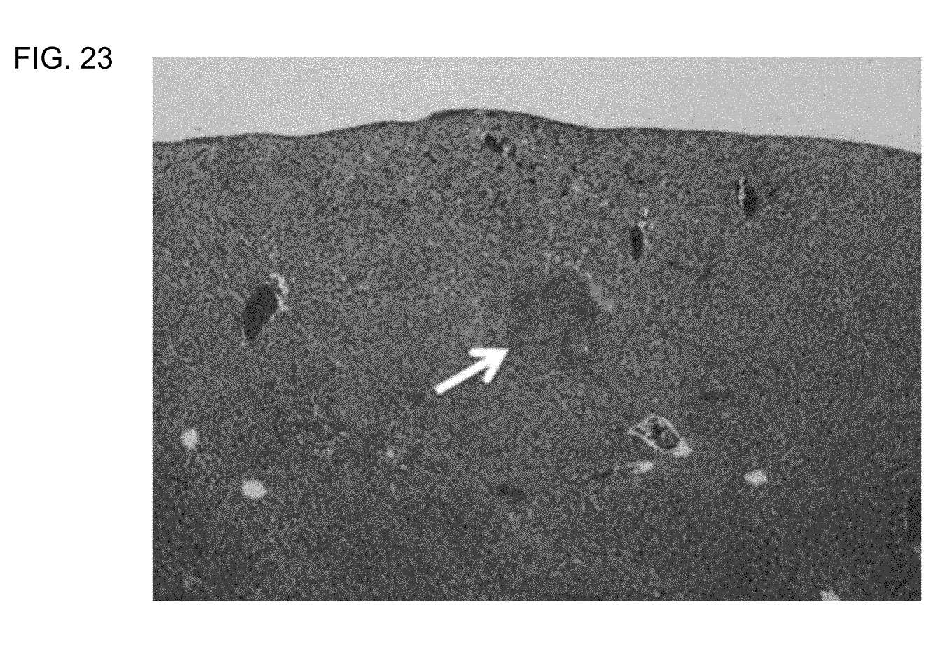

At necropsy, metastases were examined. Both macrometastases and micrometastases were scored for all metastatic evaluations. However, only the microscopic evaluations are presented here. The pancreas as well as the primary sites of metastases, including the liver, lung, and bowel, were removed and carefully sectioned for histological examination (FIG. 23). All macrometastases observed during necropsy were confirmed upon histological analysis. Additional micrometastases were found in some mice upon histological examination of the tissue sections. Each experiment was repeated at least twice.

### Development of KPC and KPCA−/− Primary Epithelial Tumor Cell Lines

Pancreatic tumors were harvested from KPC or KPCA^{-/-} mice into transport medium [RPMI 1640, penicillin (50 U/ml), streptomycin (50 μg/ml), gentamicin sulfate (10 μg/ml), and fungizone (2.5 μg/ml); Invitrogen] and placed on ice. The tumors were diced using a surgical blade, placed in prewarmed digest medium [RPMI 1640, 5% fetal bovine serum (FBS), collagenase (1500 U/ml), and hyaluronidase (1000 U/ml); Invitrogen] and incubated at 37° C. for 1 hour. After the digest, the tumor was filtered through a cell strainer (100 μm). The cells were spun at 1500 rpm for 10 min. All of the cells were plated in a 25-cm flask in primary pancreatic tumor medium [RPMI 1640, 10% FBS, 2 mML-glutamine, 1% nonessential amino acids, 1 mM sodium pyruvate, penicillin (50 U/ml), and streptomycin (50 μg/ml); Invitrogen]. Two days later, the nonadherent cells were removed, and fresh primary pancreatic tumor medium was added to the flask. When the cells reached confluence, trypsin was added to the flask for 1 min to remove the fibroblasts. The fibroblasts were transferred to a new flask, and fresh medium was added to the original flask. This procedure was repeated until pure epithelial and fibroblast cell lines were obtained.

### Cells

KPC and KPCA^{-/-} cell lines were developed as described previously. Panc02 cells are a methylcholanthrene-induced pancreatic tumor cell line derived from C57Bl/6 mice (40). All mouse pancreatic tumor cells were maintained in RPMI 1640 medium containing 10% FBS, 1 mM sodium pyruvate, 2 mML-glutamine, 1% nonessential amino acids (100×), penicillin (50 U/ml), and streptomycin (50 μg/ml) (Invitrogen) in a humidified incubator at 37° C., 5% CO₂. COS7 cells were maintained in Dulbecco's modified Eagle's medium containing 10% FBS in a humidified incubator at 37° C., 5% CO₂.

### Cell Proliferation

Cell proliferation was verified using cell counting kit-8 (CCK8). In brief, 2.5×10⁵ tumor cells were plated in a six-well plate in complete medium. For the 0-hour time point, the medium was removed and replaced with 1 ml of fresh medium along with 100 ml of CCK8 reagent (Sigma) once the cells adhered to the plate. The plate was returned to the incubator for 2 hours and read at 450 nm on a SpectraMax M3 plate reader, using Softmax Pro v. 6.3 software (Molecular Devices). This procedure was repeated at 24 and 48 hours.

### Quantitative RT-PCR

Pancreatic tumors were harvested from KPC and KPCA^{-/-} mice, flash-frozen in liquid nitrogen or optimum cutting temperature compound (OCT), and stored at −80° C. until RNA extraction was performed or slides were sectioned. RNA was extracted from flash-frozen pancreatic tissues using Trizol reagent. In brief, pancreatic tumors were diced in 1 ml of Trizol reagent and incubated at room temperature for 30 min. Chloroform was added (200 ml), and the samples were shaken vigorously for 15 s before incubation at room temperature for 2 min. Samples were spun at 12,000 rpm, and the aqueous phase was transferred to a fresh microcentrifuge tube. Isopropanol was added to the aqueous phase, and the samples were left at room temperature for 10 min. The samples were again centrifuged at 12,000 rpm, and the supernatant was removed. The RNA pellet was washed once in 75% ethanol, centrifuged at 9500 rpm, and left to air-dry for 30 min at room temperature. The RNA pellet was resuspended in 50 ml of distilled water, which was then added to a Qiagen Mini-Prep RNA extraction column. Then, RNA purification was performed according to the manufacturer's instructions (Qiagen).

RNA was extracted from the frozen sections of pancreatic tumor embedded in OCT using Ambion's RNAqueous-Micro kit according to the manufacturer's protocol. qRT-PCR was performed on an Applied Biosystems RT-PCR machine (Life Technologies). All primers were obtained from Applied Biosystems (Life Technologies). Reactions were performed using Taq MasterMix (Life Technologies). All mRNA expression values were normalized to mouse GAPDH expression values.

### Western Blot Analysis

Cells were lysed in 250 mM NaCl, 5 mM EDTA, 50 mM tris (pH 7.4), and 0.5% NP-40 containing protease inhibitors. After lysis, the lysate was spun at 15,000 rpm for 5 min. Samples boiled in SDS sample buffer containing reducing agents (Bio-Rad) were loaded and electrophoresed on a 4 to 12% bis-tris gel (Bio-Rad) for 2 hours at 120 V. The gels were transferred onto nitrocellulose membranes at 80 V for 1 hour at 4° C. The membranes were blocked in 5% bovine serum albumin (BSA) overnight at 4° C. on a shaker. Primary antibodies were added in 2.5% BSA, and the membranes were incubated at room temperature for 2 hours. The membranes were washed and then incubated with rabbit or mouse secondary antibodies against horseradish peroxidase (1:5000; GE) for 1 hour at room temperature. The membranes were again washed and then developed using enhanced chemiluminescence reagent (GE).

Western blot analysis was performed using the following primary antibodies: a rabbit polyclonal antibody against Sema3D (1:1000; Abcam), a rabbit polyclonal antibody against AnxA2 (1:000; Santa Cruz Biotechnology), a rabbit polyclonal antibody against PlxnD1 (1:1000; Novus), or a mouse polyclonal antibody against b-actin (1:500; Santa Cruz Biotechnology).

### Restoration of ANXA2 Expression in the KPCA^{-/-} Cell Line

The full-length mouse ANXA2 cDNA (wild type and Y23A) (National Center for Biotechnology Information, GenBank: BC005763.1) was amplified using the following primers: forward, GCGTCTAGAATGTCTACTGTCCACGAAATCCTG (SEQ ID NO: 1); reverse, CGCGGATCCTCAGTCATCCCCACCACACAGGT (SEQ ID NO: 2). The amplicon was purified using the QIAquick PCR Purification Kit (Qiagen) and verified by sequencing. The QIAquick Gel Extraction Kit (Qiagen) was used to purify the PCR product, and the product was then ligated into a pHIV-EGFP plasmid. The plasmid was grown in an overnight culture under ampicillin selection and was then purified using the PureLink HiPure Plasmid Maxiprep Kit (Invitrogen). To produce lentivirus expressing mouse ANXA2, 293T cells were seeded in multiple six-well plates to 80% confluence. The plasmid containing ANXA2 was cotransfected with packaging plasmids into 293T cells as previously described (41), using Lipofectamine 2000 (Invitrogen) in Opti-MEM medium. Lentiviral supernatant was collected at 48 hours. For infection, KPC cells were seeded in a 75-cm flask to 80% confluence. For each 75-cm flask, 5 ml of lentiviral supernatant was added with polybrene (5 μg/ml), and the cells were incubated for 48 hours before being harvested. The cells were then analyzed by fluorescence-activated cell sorting (FACS) for GFP-positive cells and maintained in culture medium containing puromycin (0.25 mg/ml), which is a nonselecting dose. AnxA2 abundance in the sorted cells was confirmed by Western blot.

Overexpression of Sema3D, shRNA Knockdown of Sema3D, and shRNA Knockdown of PLXND1 in KPC Cells

Lentivirus expressing mouse Sema3D cDNA (pReceiver-Lv203, GeneCopoeia), mouse Sema3D shRNA (GeneCopoeia), or mouse PLXND1 shRNA (Thermo Scientific) was produced as described earlier. For infection, KPC cells were seeded in a 75-cm flask to 80% confluence. For each 75-cm flask, 5 ml of lentiviral supernatant was added with polybrene (5 μg/ml) and incubated for 48 hours before the cells were harvested. The cells were then analyzed by FACS for GFP-positive cells. Sema3D and PlxnD1 abundance in the sorted cells was assessed by Western blot.

### Plasmid Transfection and RNA Interference

For plasmid transfection and RNA interference, cells were seeded in 10-cm dishes to 80% confluence. For each dish, 20 pmol of each siRNA duplex was transfected with Lipofectamine 2000 in serum-containing medium according to the manufacturer's instructions (Invitrogen). For invasion analysis, the culture medium was replaced with serum-free medium 24 hours after transfection, and the cells were harvested and plated in the invasion chamber 24 hours later. The ANXA2 (4), PLXND1, and scramble siGENOME siRNAs were purchased from GE.

### Microarray Analysis

RNA was extracted from the KPC and KPCA^{-/-} cell lines using the Qiagen RNA Mini Kit according to the manufacturer's instructions. Microarray analysis was performed at the Johns Hopkins Deep Sequencing and DNA Microarray Core using the Affymetrix MoEx Mouse Exon 1.0 ST array (Affymetrix). Data were extracted, RMA (robust multi-array average)-normalized, and analyzed for gene-level expression on the Partek Genomics Suite 6.6 platform (Partek Inc.). Gene Ontology analysis was performed using Spotfire DecisionSite with Functional Genomics Gene Ontology Browser (Tibco Spotfire Inc.). The genes that were increased or decreased in abundance by more than 1.5-fold were included for the analysis, which compared them to the universe of all the microarray's genes. The canonical pathways containing these genes were ranked by P values according to Fisher's exact test. The lower the P value is, the less likely these results could have occurred by chance, and thus, the more significantly the given pathway is enriched with genes that are either increased or decreased in abundance. The studies were prioritized into two functional categories (cell movement pathway and cell morphology and remodeling pathway) that are the most significantly enriched with genes increased and decreased in abundance, respectively, to examine the role of AnxA2 in invasion and metastasis. The six genes that were the most significantly increased or decreased in abundance from each of the two functional categories were selected for further validation by RT-PCR in independent KPC and KPCA^{-/-} tumor tissue.

### Immunofluorescence

OCT-embedded frozen pancreatic tumors from KPC and KPCA^{-/-} mice were sectioned and fixed in 4% paraformaldehyde for 10 min. The tumor sections were incubated in PBS containing 0.1% Triton X-100 for 5 min and then washed with PBS. Then, the tumor sections were blocked with 10% normal goat or donkey serum in PBS for 1 hour. Next, the tumor sections were incubated with antibodies against Sema3D (Abnova), PlxnD1 (Novus), Sema3A (Abcam), Snail1 (Abcam), NG2 (Chemicon), or AnxA2 (Cell Signaling) at a dilution of 1:25, 1:50 (Snail1), 1:300 (NG2), or 1:100 (AnxA2) in 10% normal goat or donkey serum overnight at 4° C. After the overnight incubation, the tumor sections were washed and were further incubated with FITC-conjugated goat antibodies against rabbit immunoglobulin G (IgG), FITC-conjugated goat antibodies against mouse IgG (Southern Biotechnology), or AF594-conjugated donkey antibodies against rabbit IgG (Life Technologies) at a 1:200 dilution or according to the manufacturer's instructions (AF594) in 10% normal goat or donkey serum at room temperature for 1 hour. NG2 staining was performed according to a previously described protocol (42). The tumor sections were subsequently washed and mounted in medium containing DAPI (4′,6-diamidino-2-phenylindole) (Vector Labs) before being examined under a fluorescence microscope.

### Sema3D ELISA

Sema3D ELISA was performed according to the manufacturer's protocol (Cusabio). In brief, KPC and KPCA^{-/-} cells were plated at 2.5×10³ cells per well in a six-well plate. The next day, the medium was replaced with fresh medium containing the indicated amount of mouse monoclonal antibody against AnxA2 (clone Z014, both human and murine AnxA2-reactive; Invitrogen), and the cells were returned to the incubator for 24 hours. After incubation, the supernatant was removed from each well and spun at 1500 rpm for 5 min to remove any floating cells. The supernatant from the KPC cells was diluted 1:66 in the sample buffer provided in the kit, whereas the supernatant from the KPCA^{-/-} cells was diluted 1:3 in the sample buffer. These dilutions were chosen because the final concentrations of Sema3D in these samples approximated 300 pg/ml, which falls in the middle of the standard curve.

### Coimmunoprecipitation

Coimmunoprecipitation of AnxA2 and Sema3D was performed as follows. The Pierce Crosslink IP Kit (Thermo Scientific) was used to cross-link AnxA2 antibodies (BD Biosciences) to beads before performing coimmunoprecipitation according to the manufacturer's instructions with modifications. In brief, Protein A/G Plus agarose beads were loaded onto a column along with 5 μg of AnxA2 antibodies. Then, the column was incubated on a rotator for 60 min at room temperature. Next, after washing the beads three times with coupling buffer followed by centrifugation, 2.5 mM disuccinimidyl suberate was added to the column, and the cross-linking reaction was allowed to proceed for 1 hour on a rotator at room temperature. Then, the column was washed three times, and antibody cross-linking was confirmed by SDS-polyacrylamide gel electrophoresis (SDS-PAGE). Next, cell lysates were added to the cross-linked antibodies, and coimmunoprecipitation was performed at 4° C. for 2 hours on a rotator. After the incubation, the column was washed, and the beads were boiled in SDS buffer containing reducing agents. Finally, coimmunoprecipitates were analyzed by SDS-PAGE followed by Western blot.

Coimmunoprecipitation of Sema3D and PlxnD1 was performed as follows. First, rabbit polyclonal antibodies against Sema3D (Abcam) or rabbit polyclonal antibodies against IgG (Abcam) (1 mg) were added to the cell lysates and incubated for 2 hours on an end-over-end rotator at 4° C. Then, 100 ml of the Protein G Sepharose 4 Fast Flow bead slurry (GE) was added to the cell lysates in lysis buffer containing 150 mM NaCl, 50 mM tris (pH 7.4), and 1% NP-40. The lysate was incubated with the beads at 4° C. overnight on an end-over-end rotator. Then, the beads were pelleted by pulse spin and washed five times (5 min each) in ice-cold lysis buffer [200 mM NaCl, 50 mM tris (pH 7.4), and 1% NP-40] on an end-over-end rotator. Finally, the beads were boiled in SDS sampling buffer containing reducing agents, and the coimmunoprecipitates were analyzed by SDS-PAGE followed by Western blot.

Coimmunoprecipitation of Sema3D and NP-1 was performed as follows. First, rabbit polyclonal antibodies against Sema3D (Abcam) or rabbit polyclonal antibodies against IgG (Abcam) (1 μg) were added to the cell lysates and incubated for 2 hours on an end-over-end rotator at 4° C. Then, 100 ml of the Protein G Sepharose 4 Fast Flow bead slurry (GE) was added to the cell lysates in lysis buffer containing 150 mM NaCl, 50 mM tris (pH 7.4), and 1% NP-40. The lysate was incubated with the beads at 4° C. overnight on an end-over-end rotator. Then, the beads were pelleted by pulse spin and washed five times (5 min each) in ice-cold lysis buffer [200 mM NaCl, 50 mM tris (pH 7.4), and 1% NP-40] on an end-over-end rotator. Finally, the beads were boiled in SDS sampling buffer containing reducing agents, and the coimmunoprecipitates were analyzed by SDS-PAGE followed by Western blot.

### Invasion Assay

Invasion assays were performed using the Trevigen 96-well invasion assay kit according to the manufacturer's instructions with modifications (Trevigen). In brief, the Transwells were coated overnight with 1× basal membrane extract, and the cells were serum-starved 24 hours before the assay. Then, the cells were plated at 5×10⁵ cells per well in triplicate in the top well of the Transwell plate. Invasion was measured 24 hours later using CCK8 (Sigma). Briefly, the cells in the top well were removed, and the wells were washed three times with the washing buffer provided in the kit. The top well of the Transwell plate was placed in a fresh 96-well plate containing 170 ml of complete cell medium and 17 ml of CCK8 reagent. The plate was returned to the incubator and incubated at 37° C., 5% CO₂ for 2 to 4 hours in the dark. After the incubation, the top chamber of the Transwell plate was removed, and the plate was read at 450 and 650 nm. Serum-free medium was added to the bottom well of the controls. CCK8 units were adjusted by subtracting the background invasion of the serum-free control from the experimental groups.

### Immunohistochemistry

Immunohistochemistry staining for Sema3D and PlxnD1 was performed using a standard protocol on an automated stainer from Leica Microsystems. After deparaffinization and hydration of tissue, heat-induced antigen retrieval was performed with EDTA buffer (pH 9.0) for 20 min. Incubation with rabbit antibodies against Sema3D (Abcam) at a 1:100 dilution or rabbit antibodies against PlxnD1 (Novus) at a 1:50 dilution for 30 min was followed by incubation with secondary antibody from the bond polymer REFINE detection kit (Leica Microsystems). The reaction was developed using the substrate 3,3′-diaminobenzidine hydrochloride (DAB; Vector Labs). All slides were counterstained with hematoxylin.

Immunohistochemistry for Ki67 and CD31 was performed manually. After deparaffinization and hydration of tissue, heat-induced antigen retrieval for Ki67 staining was performed in citrate buffer (pH 6.0) using a pressure cooker at 125° C. for 30 s and 95° C. for 10 s. Heat-induced antigen retrieval for CD31 staining was performed in EDTA buffer (pH 9.0) using a steamer for 1 hour at 97° C. Incubation with rabbit antibodies against Ki67 (Abcam) at a 1:500 dilution or rabbit antibodies against CD31 (Abcam) at a 1:100 dilution for 1 hour was followed by incubation with biotinylated secondary goat antibodies against rabbit IgG (Vector) at a 1:200 dilution for 30 min. After incubation with ABC Vectastain reagent (Vector), the reaction was developed using the DAB substrate (Vector Labs). All slides were counterstained with hematoxylin.

### AP Binding Assay

The AP binding assay was performed as previously described (25). To produce the AP fusion proteins, COS7 cells (1.5×10⁶) were transfected with 12 mg of plasmid DNA (CTRL-AP, Sema3D-AP, or Sema3E-AP), using Lipofectamine 2000 (Invitrogen) according to the manufacturer's protocol. Twenty-four hours after transfection, the culture medium was replaced with serum-free medium. Forty-eight hours after transfection, the supernatant was harvested from the cells and filtered using a 0.22-mm syringe filter. The amount of AP-tagged ligand in the supernatant was measured using a colorimetric AP assay kit (Abcam). Next, the supernatant containing the AP fusion proteins was added to COS7 cells (2.5×10⁵), which were transfected with 2 μg of PLXND1 or NP-1 per well for 48 hours in a six-well plate, for 75 min with gentle rocking at room temperature. After incubation, the cells were washed six times with HBH [1× Hanks' balanced salt solution, 0.05% BSA, 20 mM Hepes (pH 7.0), 6 mM calcium chloride, and 2 mM magnesium chloride]. Next, the cells were fixed in 60% acetone, 3% formaldehyde, and 20 mM Hepes (pH 7.0) for 1 min. Then, the cells were washed three times with HBH, and the HBH was replaced with HBS [20 mM Hepes (pH 7.0) and 150 mM NaCl]. Endogenous AP was inactivated by incubating in a humidified chamber at 65° C. for 110 min. Finally, AP was visualized using AP stain [100 mM tris (pH 9.5), 100 mM NaCl, 5 mM MgCl2, nitroblue tetrazolium (0.33 mg/ml), and 5-bromo,4-chloro,3-indolylphosphate (0.17 mg/ml)]. The stained cells were visualized under a microscope.

### TUNEL Assay

The TUNEL (terminal deoxynucleotidyl transferase-mediated deoxyuridine triphosphate nick end labeling) assay was performed according to the manufacturer's instructions (Roche).

### Inhibition of Exocytosis

KPC cells were plated to 80%0/confluence in 10-cm culture dishes. Before beginning the assay, the culture medium was replaced with fresh serum containing pancreatic tumor cell medium. GolgiPlug (brefeldin A; BD Biosciences) was added to the fresh culture medium at a concentration of 1 μl/ml of culture medium. Five hours later, the cell supernatant was removed, spun at 1500 rpm for 5 min, and frozen at −80° C. until analyzed using the Sema3D ELISA kit.

### Statistical Analysis

Statistical analysis was performed using GraphPad Prism version 6.0 software (GraphPad Software). Fisher's exact test was used to compare differences between treatment groups. Linear regression analysis was used to compare Sema3D secretion between KPC and KPCA^{-/-} cell lines after AnxA2 antibody inhibition, as well as the proliferation rates of different cell lines. Mouse survival was analyzed by the Kaplan-Meier method and the log-rank test. A P value of <0.05 was considered statistically significant.

### Example 2: ANXA2 is Essential for PDA Metastasis Formation in a Transgenic Mouse Model of PDA

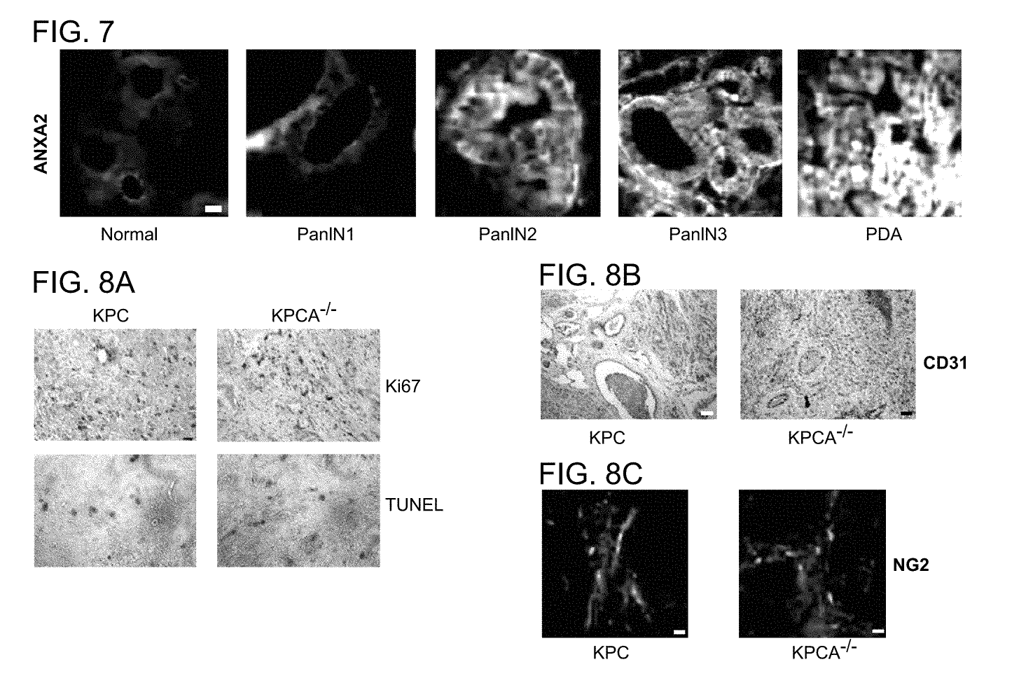

To evaluate the mechanism by which AnxA2 facilitates PDA development and metastases, KRAS^{G12D} TP53^{R172H} PDX-1-CRE^{+/+} (KPC) mice, which are genetically engineered to develop spontaneous PDA tumors (16), and ANXA2 homozygous knockout (ANXA2^{-/-}) mice were crossed to generate KPC×ANXA2^{-/-} (KPCA−/−)mice. ANXA2^{-/-} mice have a normal life span and fertility but display defects in neoangiogenesis in vivo and in ex vivo assays (17, 18). PDA development in the KPC mice recapitulates the progression from low-grade pancreatic intraepithelial neoplasms (PanINs) to invasive PDA in humans (16). It was previously reported that the abundance of AnxA2 during the course of PDA development in KPC mice and in humans also increases with PanIN progression (4). AnxA2 is localized mainly in the cytoplasm of normal pancreatic epithelial cells and in the inner luminal surface of early PanIN lesions. However, this polarity of AnxA2 distribution is changed in later-stage PanINs when AnxA2 is relocated to the outer luminal surface in PanIN2 and PanIN3 lesions. In accordance with this, AnxA2 was found on the surface of all PanIN3 and invasive PDA cells (FIG. 7).

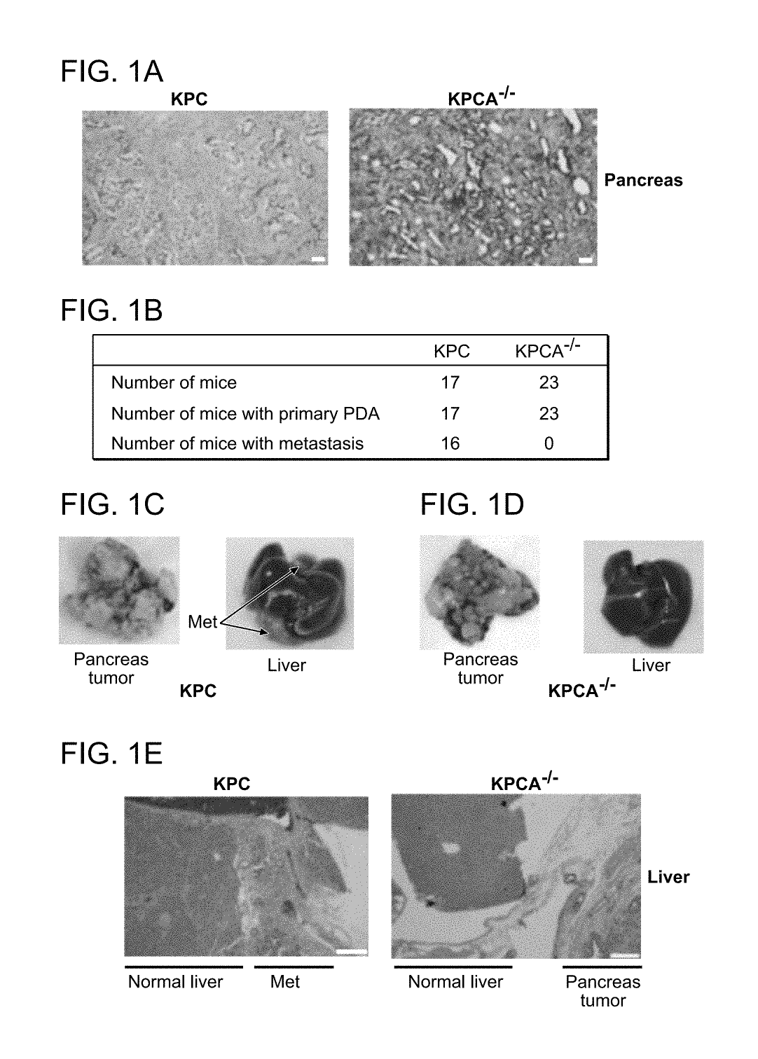

Histological analysis confirmed the presence of primary PDAs in both cohorts of mice (FIG. 1A). Both KPC and KPCA^{-/-} mice developed PanIN1 lesions at as early as 4 weeks of age. Additionally, both cohorts of mice developed PanIN2 and PanIN3 lesions at as early as 8 and 10 weeks of age, respectively. By 3 months of age, roughly 75% of mice in both cohorts had PanIN3 lesions, and by 4 months, an average of 65% of mice in both cohorts had histologically confirmed PDA when the mice were euthanized (Table 4).

TABLE 4
Primary tumor development was compared between
mice expressing ANXA2 (KPC) and mice lacking ANXA2
expression (KPCA^{-/-}).

| | KFC | KPCA^{-/-} | |
|---|---|---|---|
| PanIN3 (≥3 months) | 14/19 (73.7%) | 22/22 | (100%) |
| Invasive PDA (≥4 months) | 8/13 (61.5%) | 11/16 | (68.6%) |

PanIN—pancreatic intraepithelial neoplasia;
PDA—pancreatic ductal adenocarcinoma

In addition, primary tumors derived from KPC and KPCA^{-/-} mice demonstrated similar rates of proliferation and apoptosis (FIG. 8A). All KPC and KPCA^{-/-} mice eventually died from the growth of primary PDA as previously observed with KPC mice (16); thus, there was also no observable difference in survival. However, despite these similarities in primary tumor growth between the KPC and KPCA^{-/-} mice, the metastatic potential of PDA tumors differed between the two cohorts of mice: Upon gross examination of the mice in both cohorts, metastatic lesions were observed in the liver, peritoneal cavity, and lungs of 16 of 17 KPC mice (FIG. 1B and FIG. 1C), whereas no observable gross metastatic lesions were seen in 23 KPCA−/− mice (FIG. 1B and FIG. 1D). Despite the ability of primary PDA tumors to grow relatively close to the liver in KPCA−/− mice, only primary PDA tumors expressing ANXA2 in KPC mice were able to invade and grow into

the liver (FIG. 1E).

Because the function of AnxA2 in angiogenesis may play a role in controlling metastatic formation, examined the vascular network in PDAs from KPC and KPCA^{-/-} mice was examined. No obvious differences were observed in the tumor vascular networks between KPC and

KPCA^{-/-} mice, as characterized by immunohistochemistry of the endothelial cell marker CD31 (FIG. 8B) and the pericyte marker NG2 (FIG. 8C), suggesting that the function of AnxA2 in angiogenesis is unlikely to mediate its role in PDA metastasis.

### Example 3: Reintroduction of ANXA2 Restores the Metastatic Potential of ANXA2^{-/-} PDA Cells

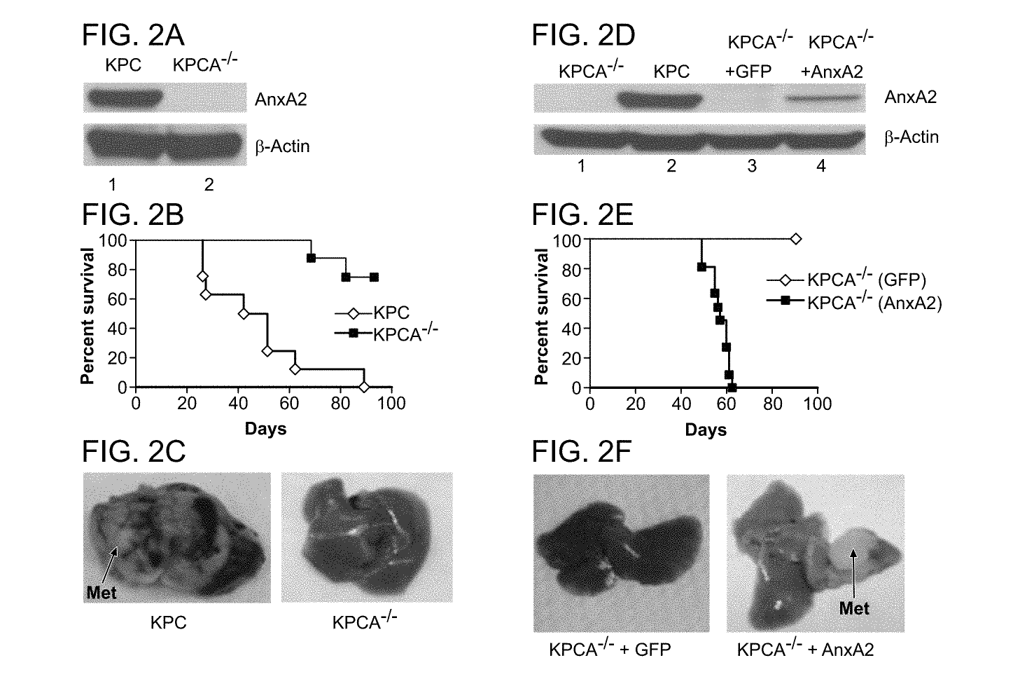

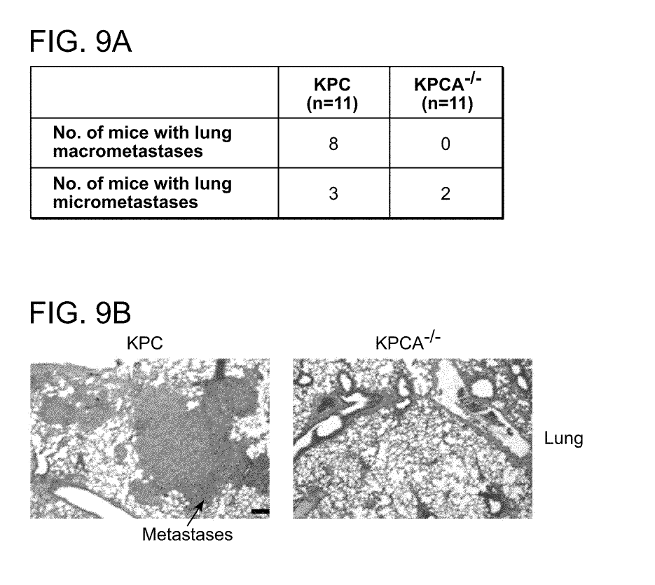

Next, it was investigated whether it was specifically the ANXA2 deficiency or additional genetic alterations that led to the loss of metastatic potential in the PDA cells in KPCA^{-/-} mice. To address this question, cell lines were established from the primary tumors of KPC and KPCA^{-/-} mice to be used in a previously reported liver metastasis model in which cells were injected into the circulation via the spleen (4, 19). Western blot analysis confirmed that the cell line established from a KPCA^{-/-} mouse had no detectable AnxA2 abundance, whereas the cell line established from a KPC mouse did (FIG. 2A). The KPC and KPCA^{-/-} cell lines were then injected into the hemi-spleens of syngeneic mice, which were assessed for survival and liver colonization, over the course of, at most, 90 days. Most (8 of 10) of the mice that received an injection of KPCA^{-/-} cells survived to the end of the 90-day study (two mice died as a result of tumors that formed at the splenic injection site) and none developed liver nodules (FIG. 2B and FIG. 2C). In contrast, all mice that received an injection of KPC cells developed liver nodules and, accordingly, had relatively decreased survival (FIG. 2B and FIG. 2C). In addition, it was identified that KPCA^{-/-} cells were rarely able to form micrometastases and did not form colonies in the lung (FIG. 9A and FIG. 9B).

Because the above experiment showed that KPCA^{-/-} cells injected into the hemi-spleen of syngeneic mice were unable to colonize the liver, it was next investigated whether the restoration of ANXA2 expression would enable KPCA^{-/-} cells to colonize the liver. Full-length ANXA2 complementary DNA (cDNA) was introduced into KPCA^{-/-} cells in culture by infection with a green fluorescent protein (GFP)-encoding lentivirus, and the cells were sorted by GFP expression. Although the expression amounts achieved were only ˜25% of the endogenous amounts of AnxA2 in KPC cells (FIG. 2D), the transduced cells were able to colonize the liver and cause decreased survival in all mice that received a splenic injection of AnxA2-restored KPCA^{-/-} cells (FIG. 2E and FIG. 2F). Thus, AnxA2 has a major role in metastatic PDA colonization in this mouse model.

### Example 4: The Expression of Sema3D and PLXND1 is Differentially Regulated in Pancreatic Tumors from KPC Versus KPCA^{-/-} Mice

Next, the KPC and KPCA^{-/-} cell lines were used to investigate the downstream pathways that mediate the function of AnxA2 in PDA metastasis formation. A comprehensive mRNA expression profile comparing KPC and KPCA^{-/-} cells using microarray gene expression analysis followed by Spotfire Gene Ontology Browser analysis revealed the top four gene functional categories that were enriched with genes of increased abundance and the top five gene functional categories that were enriched with genes of decreased abundance (Table 1).

TABLE 1
Functional assignment of gene expression changes in KPCA−/− mice.
The number of genes in each subset of functional categories that are
increased or decreased in abundance in KPC versus KPCA^{-/-} primary
pancreatic tumor cell lines.

| | No. of genes with fold | | No. of genes with fold |
|---|---|---|---|
| | change >1.5/total | | change >1.5/total |
| | genes in the set* | | genes in the set* |
| Call movement | 54/433 | Cell morphology | 8/22 |
| | 46/277 | and remodeling | 20/97 |
| | | | 15/68 |
| TGF-β pathway | 55/394 | Adhesion | 14/58 |
| | | | 17/77 |
| Signaling | 39/293 | Cell movement | 55/433 |
| | 47/369 | | 43/277 |
| | | Cell cycle | 23/112 |
| Differentiation | 10/36 | TGF-β pathway | 32/394 |
| and development | 44/281 | | |
| | | Signaling | 49/293 |
| | | | 26/166 |

Asterisk denotes that if more than two gene sets in the same functional categories were identified, the two gene sets with the most significant P values are shown here.

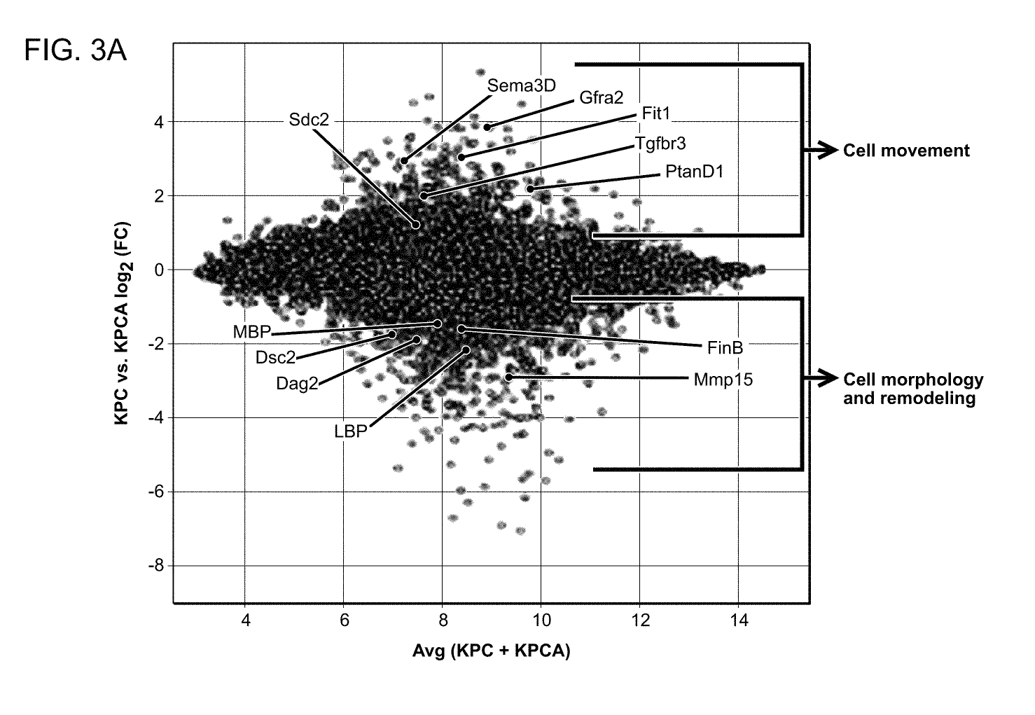

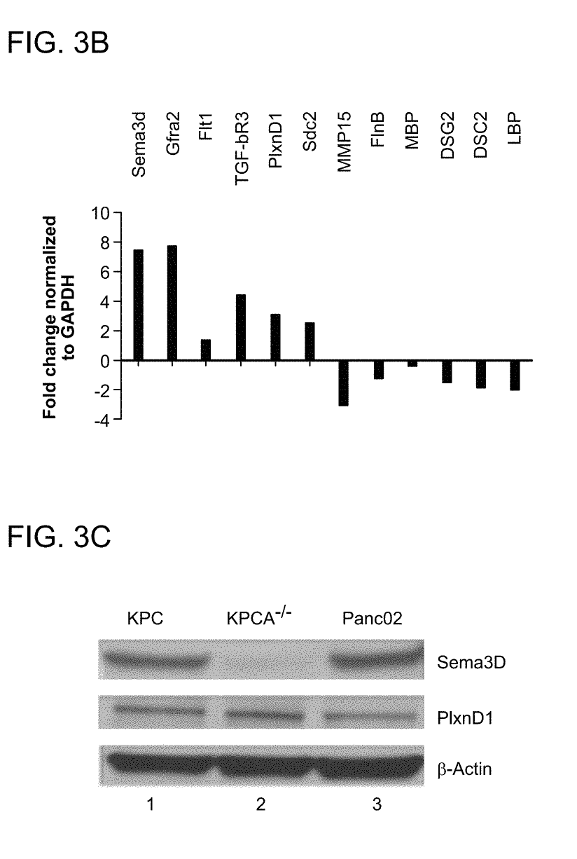

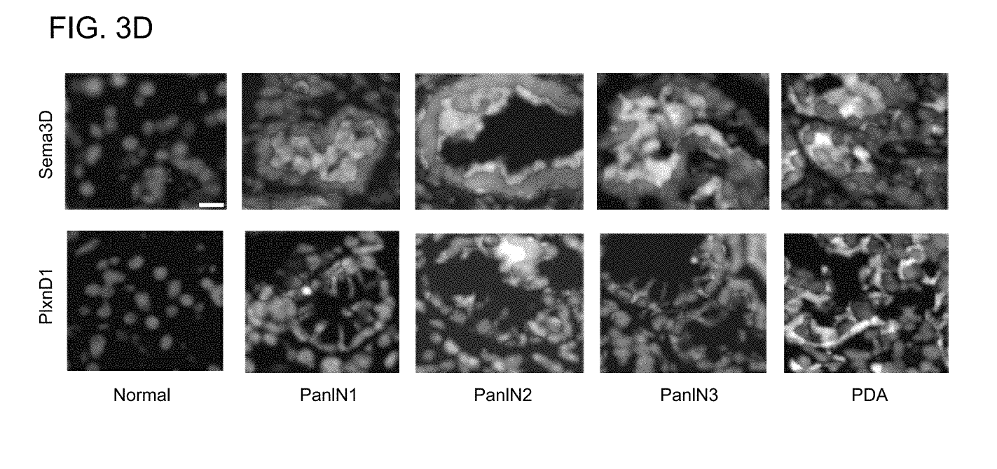

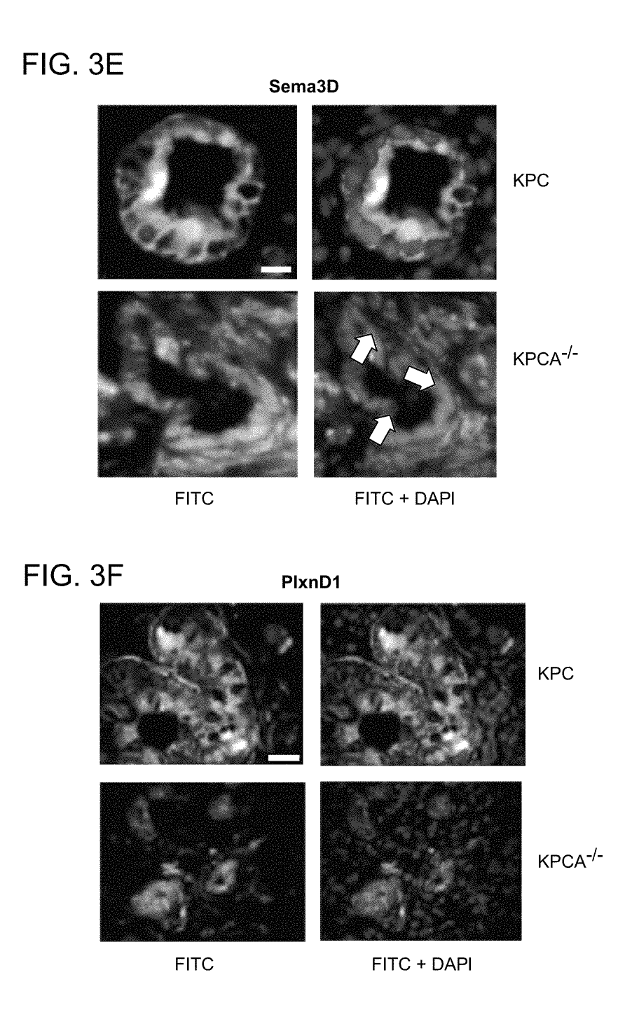

The two functional categories (cell movement pathway and cell morphology and remodeling pathway) that were the most significantly enriched with genes of increased abundance and decreased abundance, respectively, were prioritized in these studies because of their involvement in invasion and metastasis. Six genes that were the most significantly increased or decreased in abundance from each of the two functional categories were selected for further validation (FIG. 3A).

To validate the differential expression of these 12 selected genes in PDA, their expression was analyzed in additional pancreatic tumors obtained from KPC and KPCA^{-/-} mice by quantitative real-time reverse transcription polymerase chain reaction (qRT-PCR). The mRNA expression differences between the KPC and KPCA^{-/-} tumors were largely consistent with the microarray analysis of the cell lines (FIG. 3B). Sema3D and PLXND1 were of particular interest because they both belong to gene families that are frequently amplified and mutated in human PDA (5). In addition, both genes were decreased in abundance in the absence of ANXA2 (FIG. 3B), and both the class 3 Semaphorin and plexin protein families were previously shown to have a ligand-receptor relationship. As shown in the Western blot analysis, the protein abundance of Sema3D was decreased in KPCA^{-/-} cells compared to that in KPC cells, in accordance with the qRT-PCR results. However, the protein abundance of PlxnD1 was similar in both KPC and KPCA^{-/-} cells (FIG. 3C).

### Example 5: Sema3D is Aberrantly Localized in PDA from KPCA^{-/-} Mice

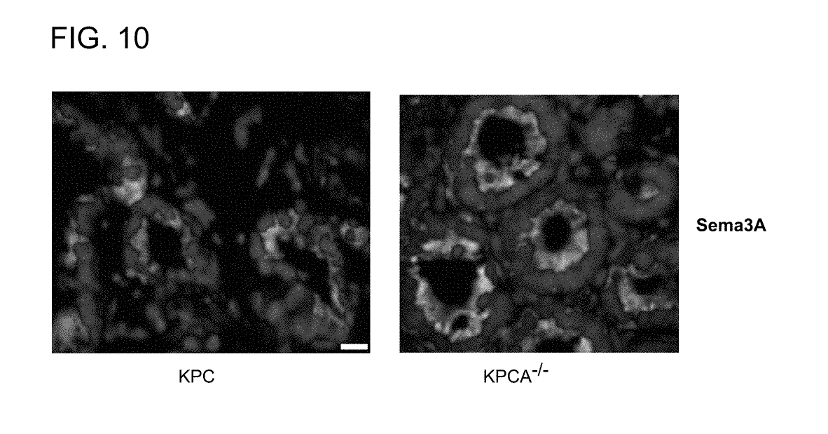

Next, the cellular localization of Sema3D and PlxnD1 were examined by immunofluorescence staining. Immunofluorescence staining of pancreatic tissue from KPC mice demonstrated that both Sema3D and PlxnD1 were less detectable in normal pancreatic tissues but gradually became more abundant in PanINs and were highly abundant in invasive PDA (FIG. 3D). In KPC mice, Sema3D was primarily present in the cytoplasm and membrane of pancreatic tumor cells and was also present on the extracellular surface, possibly in a secreted form, whereas in KPCA^{-/-} mice (which lacked ANXA2 expression), Sema3D was modestly decreased in abundance, and its presence was localized primarily to the perinuclear or nuclear regions of PDA tumor cells (FIG. 3E). In contrast, the localization of PlxnD1 and the localization of Sema3A, another member of the class 3 Semaphorin family, were unaffected by ANXA2 expression (FIG. 3F and FIG. 10).

### Example 6: AnxA2 Regulates the Secretion of Sema3D from PDA Cells

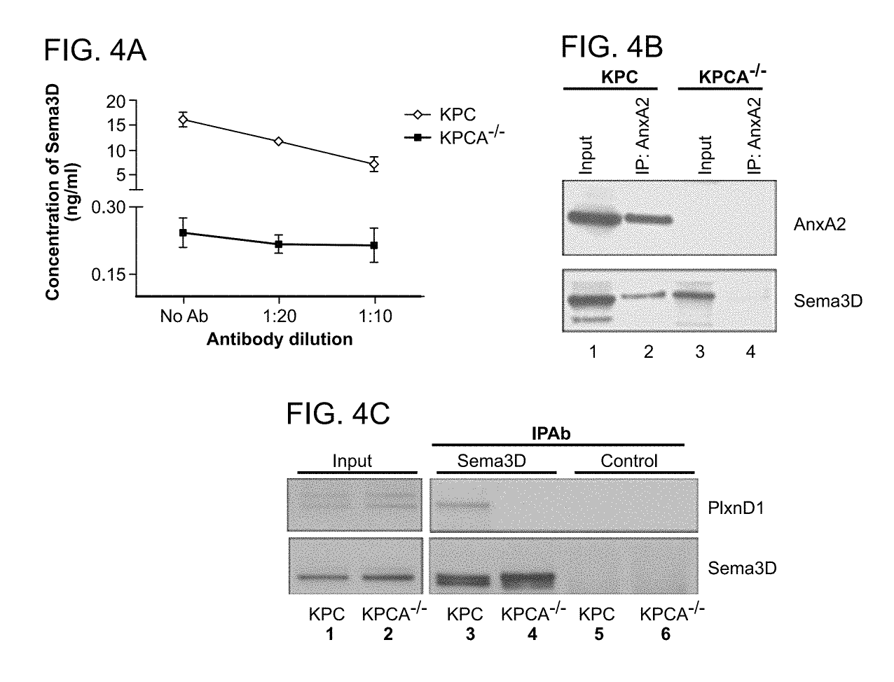

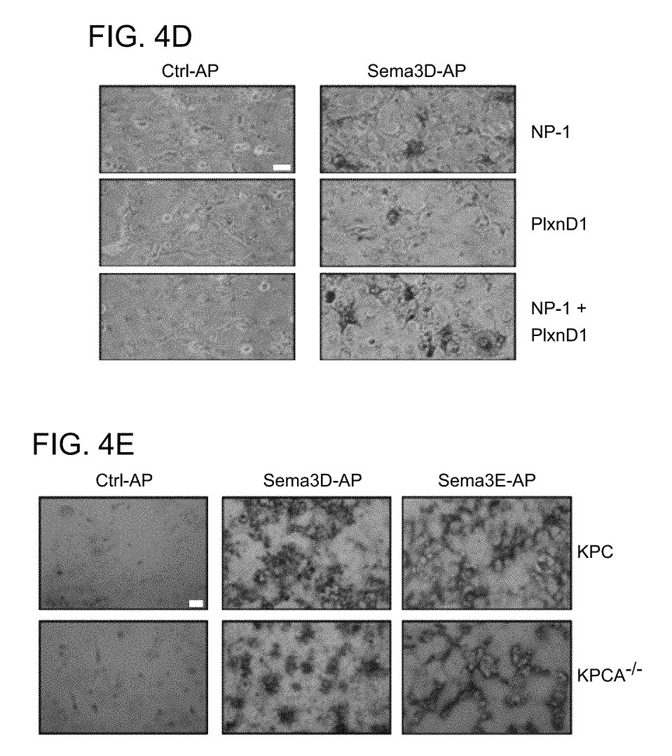

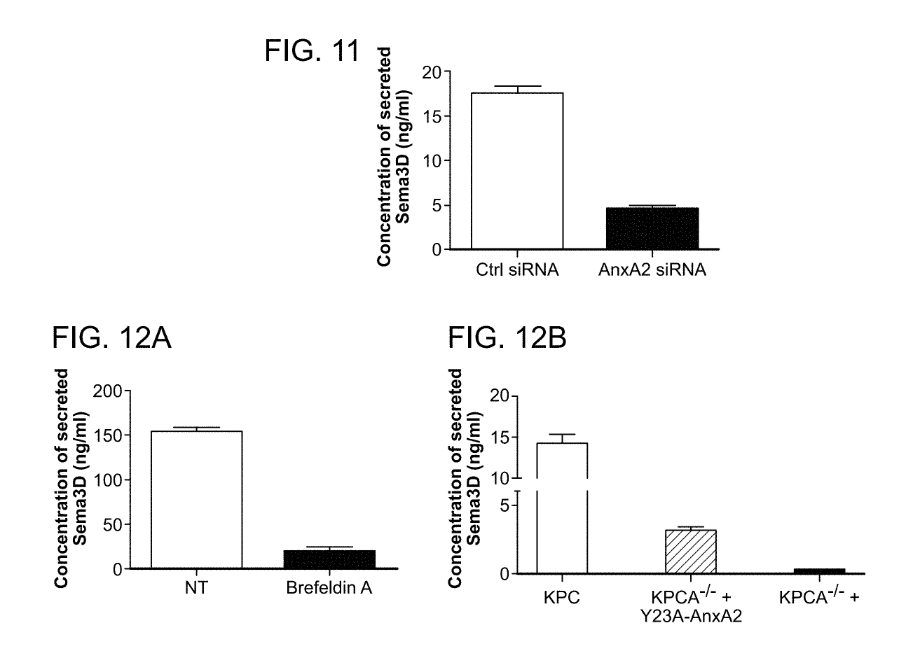

Class 3 Semaphorins, including Sema3D, are secreted proteins (20). The results thus far showed that tumor cells from KPCA^{-/-} mice have decreased abundance of Sema3D compared to KPC cells that express ANXA2, but the extracellular Sema3D, which is possibly a secreted form of Sema3D, was also substantially decreased in these cells. Thus, it was hypothesized that AnxA2 regulates the secretion of Sema3D rather than the expression of Sema3D in PDA cells. To test this hypothesis, enzyme-linked immunosorbent assay (ELISA) was used to measure the amount of secreted Sema3D in the cell culture supernatant, and found that the 24-hour secretion of Sema3D from KPCA^{-/-} cells was significantly lower than that from KPC cells (FIG. 4A). This about 70-fold decrease in Sema3D secretion cannot be explained by the decrease in protein or RNA expression of Sema3D in KPCA^{-/-} compared with KPC cells, because RNA expression was reduced by less than 10-fold (FIG. 3C). Rather, this result suggests that, in the absence of ANXA2, not only were the RNA and protein abundances of Sema3D decreased but also—and perhaps more importantly—the secretion of Sema3D was diminished. To confirm that AnxA2 mediated Sema3D secretion, it was examined whether blockade of AnxA2 with function-blocking antibodies against AnxA2 (4) could suppress Sema3D secretion. It was identified that the addition of AnxA2 antibodies to KPC cells suppressed Sema3D secretion from these cells in a dose-dependent manner, whereas Sema3D secretion from KPCA^{-/-} cells was unaffected (FIG. 4A). Similarly, small interfering RNA (siRNA) knockdown of ANXA2 in KPC cells resulted in decreased Sema3D secretion (FIG. 11). Therefore, these data support a role for AnxA2 in regulating the secretion of Sema3D from PDA cells.

To further understand the mechanism by which AnxA2 regulates the secretion of Sema3D, it was examined whether an exocytosis inhibitor could inhibit the secretion of Sema3D because AnxA2 is known to play a role in exocytosis (21). It was identified that Sema3D secretion was inhibited in the presence of an exocytosis inhibitor (FIG. 12A). The phosphorylation of Tyr²³ in AnxA2 is important for the endocytic and exocytic functions of AnxA2 (22). It was identified that a Y23A mutant of ANXA2, which has been previously reported to suppress PDA invasion and metastasis (4), largely inhibited the secretion of Sema3D from PDA cells (FIG. 12B). Thus, Sema3D secretion may be mediated by the role of AnxA2 in exocytosis.

### Example 7: AnxA2 Interacts with Sema3D and Controls the Complex Formation Between Sema3D and PlxnD1

To understand how AnxA2 mediates the secretion of Sema3D, the protein-protein interaction between Sema3D and AnxA2 was examined in PDA cells. Sema3D coimmunoprecipitated with AnxA2 in PDA cells (FIG. 4B), indicating that AnxA2 binds to Sema3D. Because AnxA2 is localized to the extracellular cell surface and is involved in exocytosis (21, 23, 24), AnxA2 may carry Sema3D to the cell surface for secretion. The secreted form of Sema3D binds neuropilin 1 (NP-1) on the surface of mammalian cells (25), and the plexin family of proteins can act as co-receptors for Semaphorins along with NP-1 by providing an intracellular domain to mediate intracellular signaling (26). However, the exact co-receptor for Sema3D is unknown. Therefore, it was hypothesized that Sema3D, secreted from PDA cells, binds to the PlxnD1 co-receptor on the surface of PDA cells in an autocrine fashion. Supporting this hypothesis, coimmunoprecipitation assays indeed showed that PlxnD1 formed a complex with Sema3D in KPC PDA cells that express ANXA2 (FIG. 4C). However, even though greater quantities of cell lysate from KPCA^{-/-} cells were used for the coimmunoprecipitation assay to study equivalent amounts of both Sema3D and PlxnD1 proteins as was isolated from KPC PDA cells, PlxnD1 was unable to be coimmunoprecipitated from the KPCA^{-/-} PDA cells, using antibodies against Sema3D. This result suggests that AnxA2 is required for Sema3D and PlxnD1 to form a complex, likely through controlling the secretion of Sema3D from PDA cells to facilitate the subsequent interaction between Sema3D and PlxnD1 on the surface of the tumor cell. Thus, in KPCA^{-/-} cells lacking ANXA2 expression, where Sema3D secretion is diminished, no Sema3D would bind to PlxnD1 on the surface of the cell.

### Example 8: Exogenous Sema3D can Bind to PlxnD1 on the Surface of the Cell

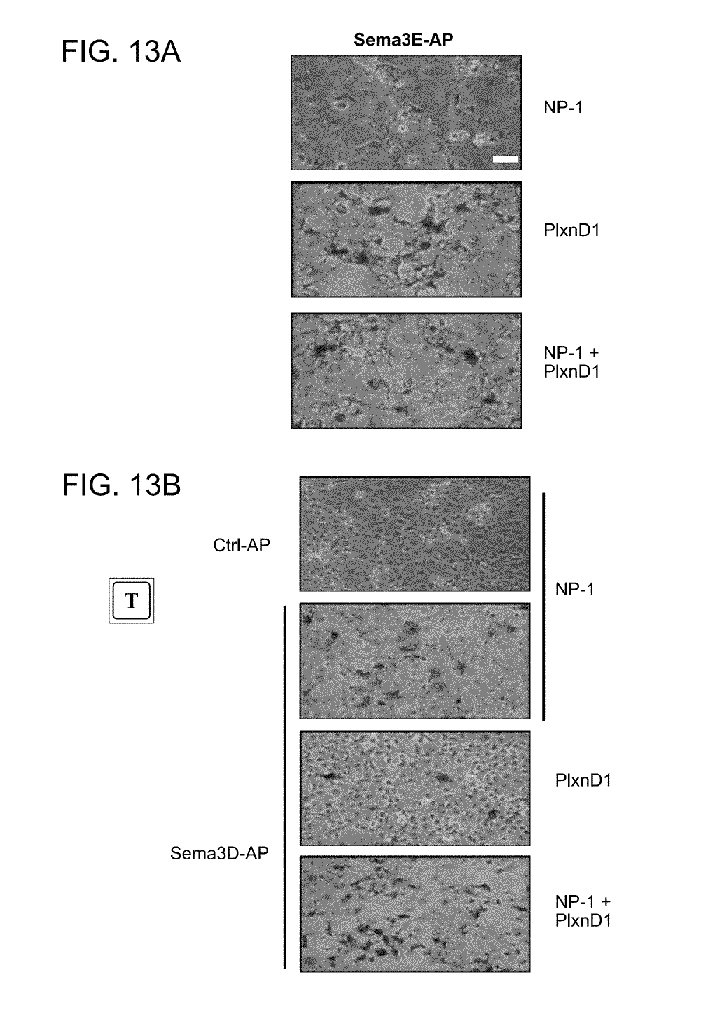

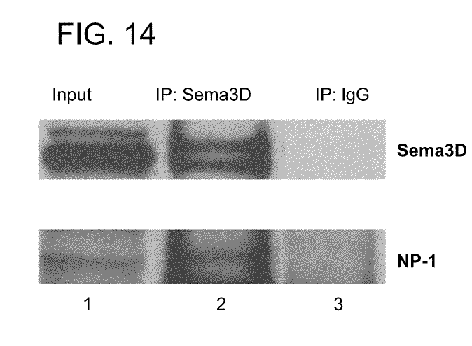

To further demonstrate that Sema3D can bind to the cell surface of PDA cells via PlxnD1, an alkaline phosphatase (AP) binding assay was performed, which was previously used to study the binding between Semaphorins and plexins on mammalians cells (25). Sema3E-AP was used as a positive control for binding to PlxnD1 in the absence of NP-1, and NP-1 was used as a positive control for Sema3D-AP binding, as described previously (FIG. 13A) (25). Sema3D-AP weakly and infrequently bound to the surface of COS7 cells transfected with a PLXND1-VSV plasmid but not to untransfected COS7 cells (FIG. 4D and FIG. 13B). Nevertheless, stronger binding of Sema3D was observed on cells cotransfected with PLXND1 and NP-1. It was further confirmed the binding of Sema3D to NP-1 by coimmunoprecipitation in PDA cells (FIG. 14). Because COS7 cells express ANXA2 (27), KPC and KPCA^{-/-} cells were also used in the AP binding assay to determine if AnxA2 is required for secreted Sema3D to bind to PlxnD1. Sema3D-AP bound PlxnD1 in both KPC and KPCA^{-/-} cells (FIG. 4E). Together, these results indicate that AnxA2 promotes the secretion of Sema3D and that Sema3D, once secreted, binds PlxnD1 independently of AnxA2.

### Example 9: Knockdown of Sema3D Decreases the Invasion and Metastatic Capacity of PDA Cells and Prolongs the Survival of PDA-Bearing Mice

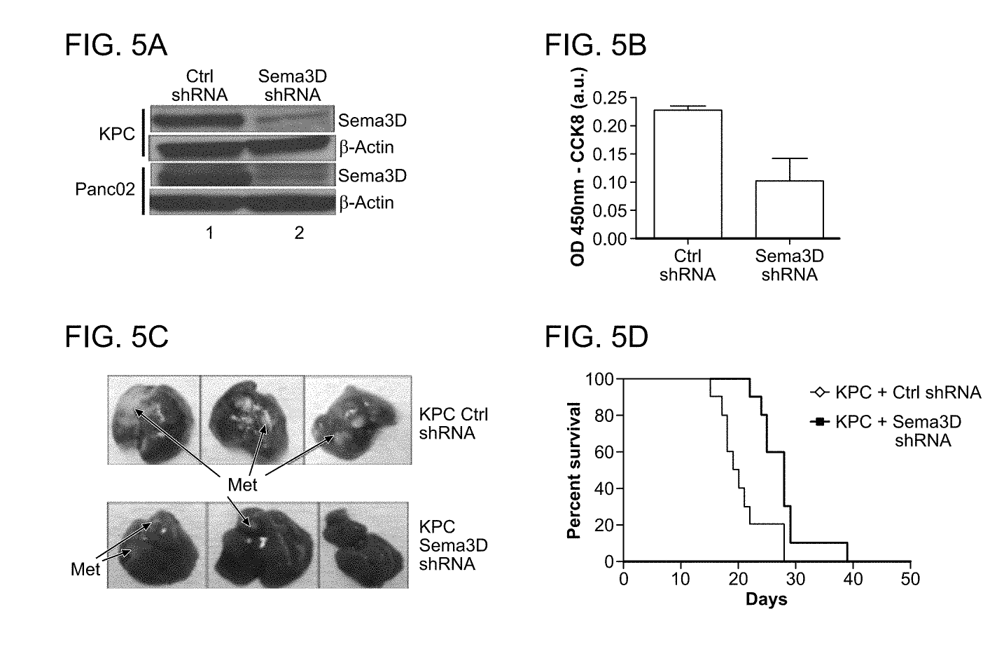

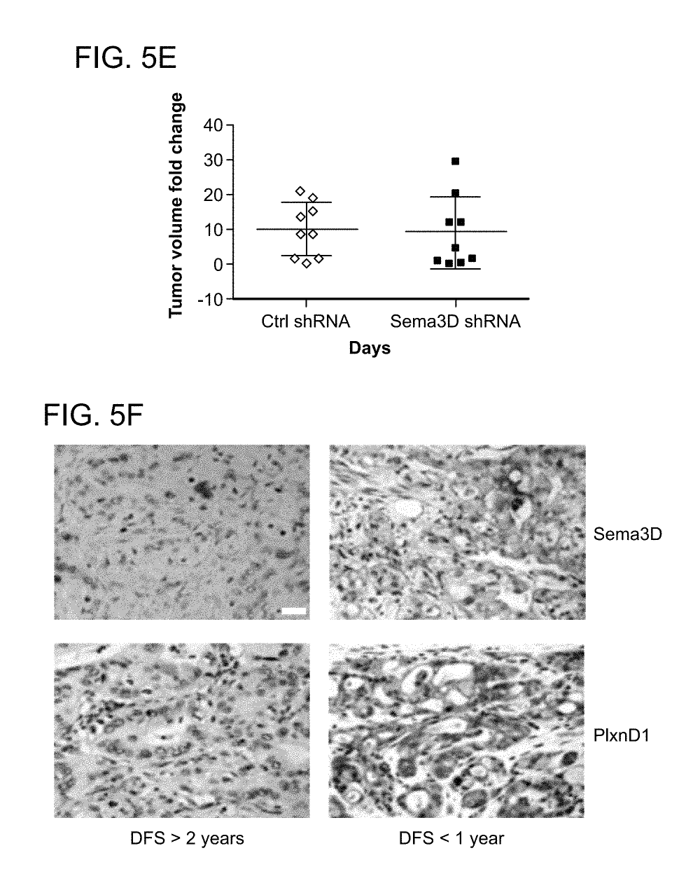

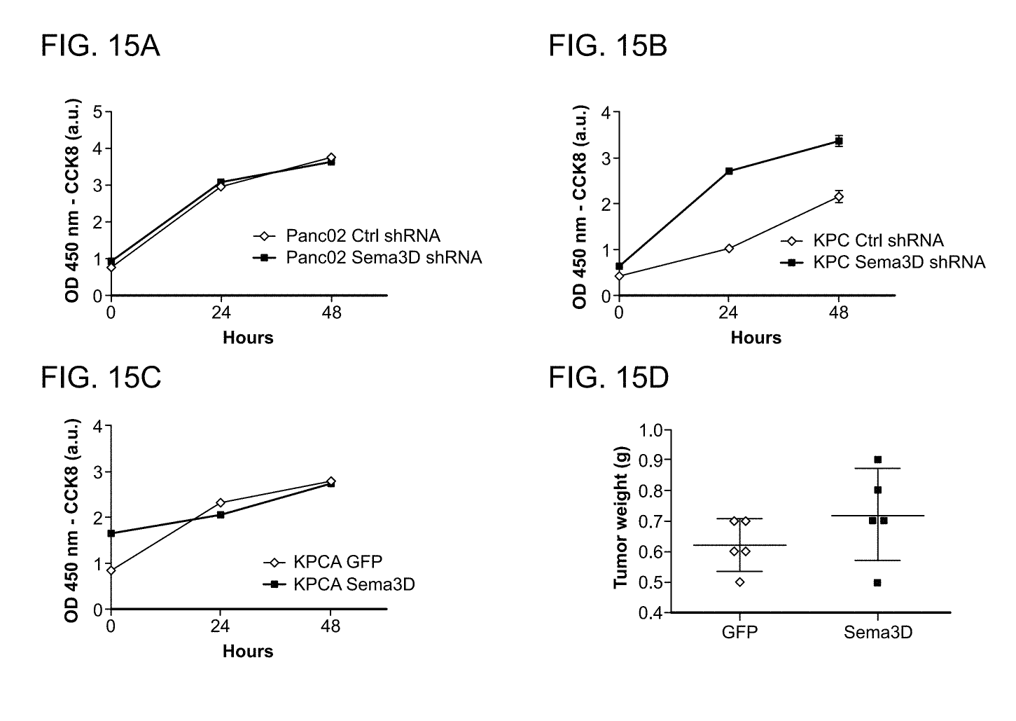

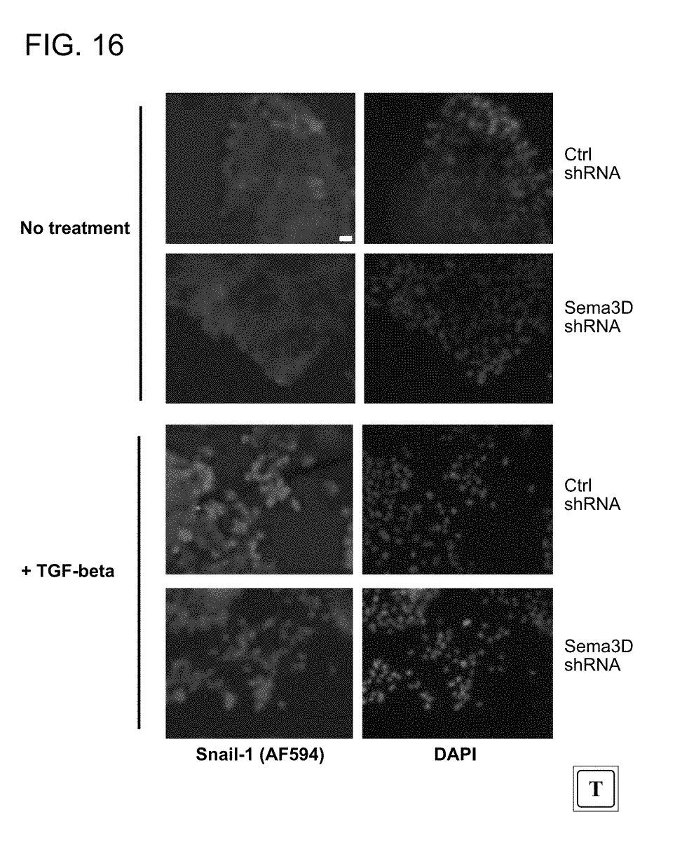

AnxA2 was previously shown to be required for PDA invasion and migration (4). The findings presented herein show that it also controls the secretion of Sema3D and, subsequently, the interaction between Sema3D and PlxnD1. Because both Sema3D and PlxnD1 are involved in cell motility (26), it was examined whether Sema3D is also involved in PDA invasion and metastasis formation. To first test this in vitro, Sema3D expression was knocked down with shRNA in the carcinogen-induced Panc02 PDA cells (FIG. 5A). The KPC cells were not used because of their leakage through the 8-mm filter in the Boyden invasion assay chamber. The invasion capacity of Panc02 cells was significantly decreased after Sema3D knockdown in this in vitro invasion assay (FIG. 5B). The low invasive activity of PDA cells with the Sema3D-targeting shRNA was not due to a decrease in proliferation (FIG. 15A and FIG. 15B). In addition, nuclear localization of Snail-1, an EMT marker and a downstream effector of PlxnD1 (15), was decreased in Sema3D knockdown cells in response to transforming growth factor-β [TGF-0, an inducer of EMT (4)] (FIG. 16), further suggesting that the role of Sema3D in PDA invasion and EMT-associated migration is likely mediated by PlxnD1.

Next the hemi-spleen liver metastasis model was used to determine whether knockdown of Sema3D expression in KPC cells results in inhibition of the metastatic potential of KPC cells in vivo. KPC cells infected with lentivirus carrying the Sema3D-targeting shRNA or those infected with control lentivirus were injected into the hemi-spleens of C57Bl/6 mice. Two weeks after tumor implant, extensive metastases were visualized in the livers of 11 of 12 mice receiving KPC cells infected with lentivirus carrying the control shRNA, whereas only small metastases were observed in 5 of 13 mice receiving KPC cells infected with the Sema3D-targeting shRNA (FIG. 5C). Moreover, an independent experiment indicated that mice receiving KPC cells with the Sema3D-targeting shRNA survived significantly longer than mice receiving KPC cells with the control shRNA FIG. 5D). These results suggest that Sema3D is required for the homing and colonization steps of PDA metastasis.

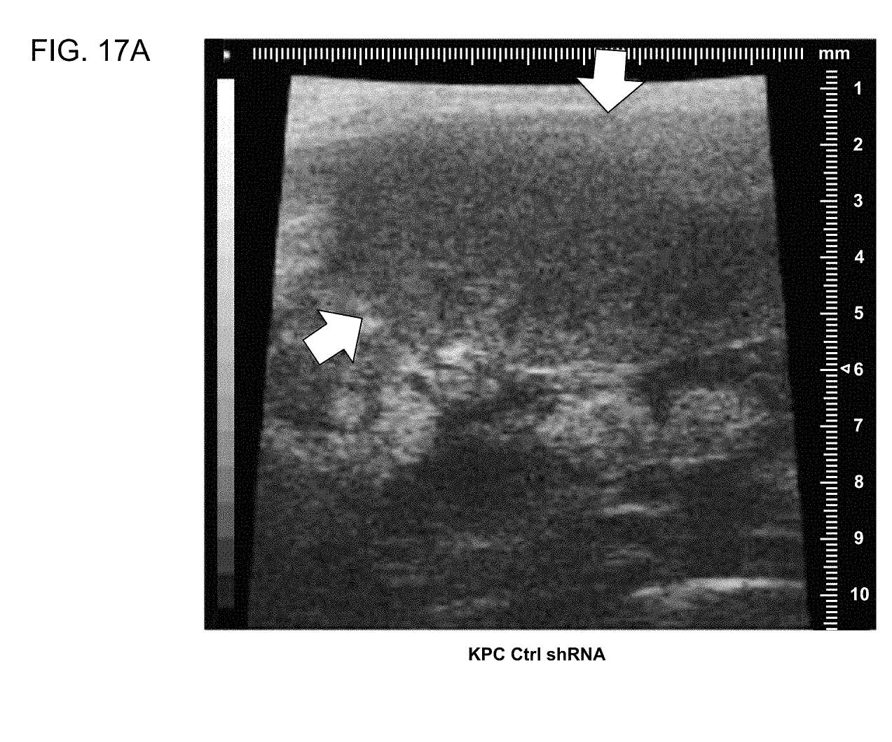

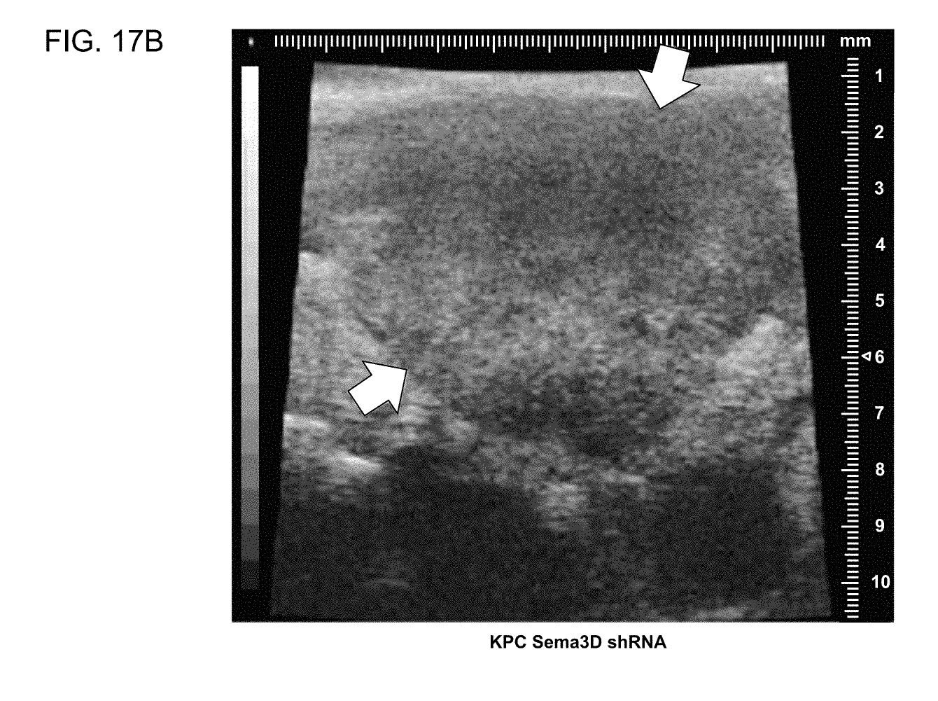

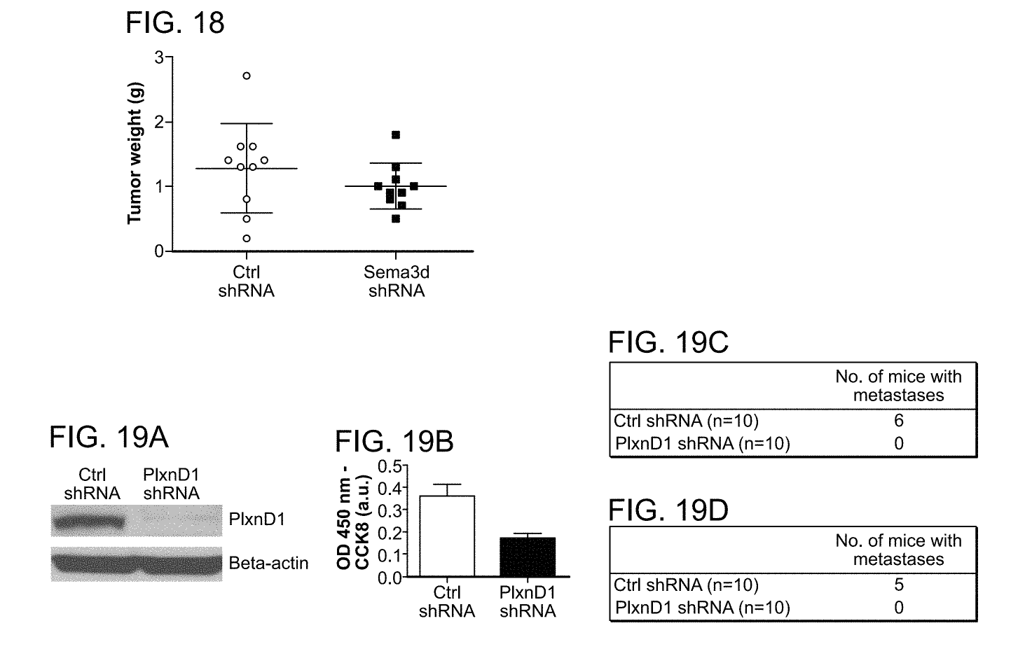

To confirm a role of Sema3D in metastasis formation, the growth rates of KPC cells with Sema3D shRNA and KPC cells were compared with control shRNA when KPC tumors were orthotopically implanted in the pancreas of syngeneic mice. Ultrasonic measurement of orthotopically implanted pancreatic tumors on days 6 and 20 after tumor implantation showed no significant difference in tumor development or growth rate between the tumors formed by KPC cells with Sema3D shRNA and those formed by KPC cells with control shRNA (FIG. SE and FIG. 17A and FIG. 17B). Furthermore, the tumor weights upon necropsy were not significantly different between the control shRNA and Sema3D shRNA groups (FIG. 18). In the control group of 10 mice, nine metastases were identified in the lung, liver, or peritoneum. However, in nine mice bearing tumors with Sema3D-targeting shRNA, only two metastases were identified (Table 2). Therefore, Sema3D does appear to have a role in controlling PDA invasion and metastasis formation. Similar approaches also demonstrated a role of PlxnD1 in PDA invasion and metastasis formation (FIG. 19A to FIG. 19D).

TABLE 2
Metastasis formation in mice after orthotopic implantation
of either KPC tumor cells with control shRNA or
KPC tumor cells with Sema3D shRNA.

| shRNA in KPC tumor cells | No. of mice with metastases |
|---|---|
| Control shRNA (n = 10 mice) | 9 |
| SEMA3D shRNA (n = 9 mice) | 2 |

P = 0.006, Fisher's exact test.

### Example 10: Sema3D Abundance is Associated with Metastasis Formation in Human PDA

To further establish the role of Sema3D in PDA metastasis formation, Sema3D immunohistochemistry was performed on human PDA tissue specimens. About 50% of surgically resected human PDAs had abundant Sema3D (in >50% of the tumor cells), whereas the remaining 50% of PDAs expressed low amounts of Sema3D (<5% of tumor cells) (FIG. 5F). Resected PDAs presenting with abundant Sema3D were observed in 15 of 20 patients (75%) with a disease free survival (DFS) of <1 year, compared to only 4 of 15 patients (26.7%) with a DFS of >2 years (FIG. 5F), suggesting that Sema3D abundance in PDA is significantly associated with early recurrence after surgical resection. In all PDAs examined, Sema3D abundance positively correlated with PlxnD1 abundance, suggesting that Sema3D and PlxnD1 may be co-regulated (FIG. 5F). Using a unique human PDA tissue bank that contains PDA specimens obtained from a rapid autopsy program, it was identified that 3 of 13 patients (23.1%) that died with local or oligometastatic disease had primary PDA tumors with abundant Sema3D, 14 of 22 patients (63.6%) with widely metastatic disease demonstrated abundant Sema3D in their primary PDA, and 17 of 22 patients (77.3%) with widely metastatic disease demonstrated abundant Sema3D in their metastatic tumors (Table 3). These results suggest that Sema3D is preferentially enriched in metastatic tumors of PDA and in primary PDAs from patients that have a poor prognosis or patients who died with widely metastatic disease.

TABLE 3
Sema3D positivity in patient primary and metastatic tumors.
The percentage of patients with Sema3D present in their primary
tumors and metastatic sites by disease status at their time of death.

| | Local disease | | |
|---|---|---|---|
| Disease status at the time | or oligometastatic | Widely metastatic | |
| of death | disease | disease | |
| Percentage of patients whose | 23.1% | 63.6% | |
| primary tumors produce Sema3D | (3 of 22 patients) | (14 of 22 patients) | |
| Percentage of primary tumors | | Primary tumor | Metastatic site |
| or metastatic sites producing | | 63.6% (14 of 22) | 77.3% (17 or 22) |
| Sema3D in patients who died | | | |
| with widely metastatic disease | | | |

### Example 11: Overexpression of Sema3D Partially Reverses the Defect in Invasion and Metastasis Formation in ANXA2-Deficient PDA Cells

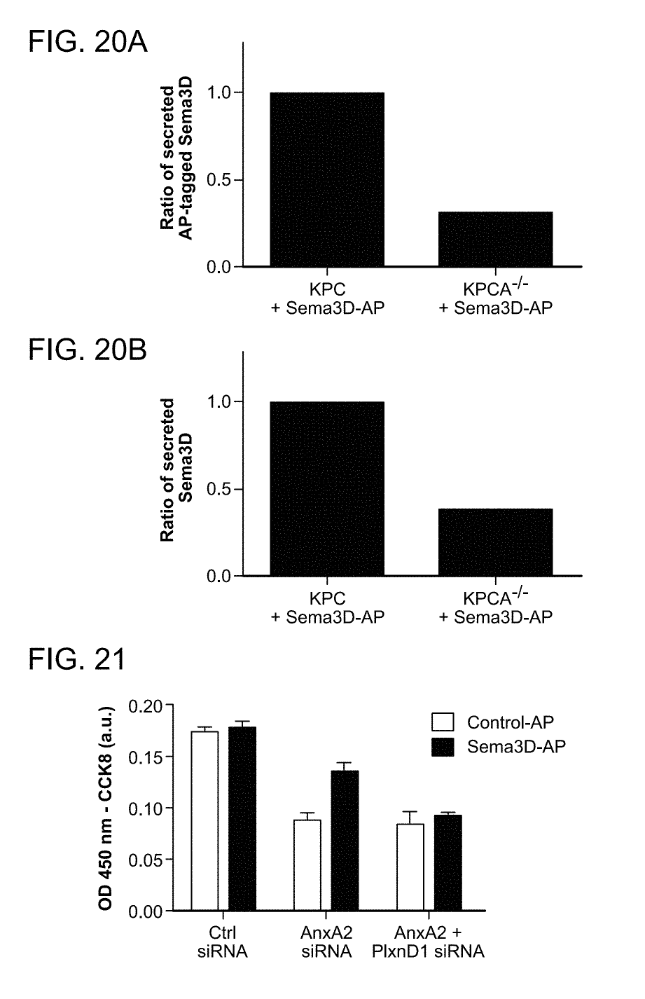

If Sema3D mediates the role of Anxa2 in PDA Invasion and metastasis formation, AnxA2-independent secretion of Sema3D may restore or partially restore the defect of ANXA2-deficient PDA cells in invasion and metastatic potential. To test this hypothesis, ANXA2 from Panc02 cells was knocked down with ANXA2-targeting siRNA as described previously (4) and concurrently transfected the cells with a plasmid constitutively overexpressing Sema3D through a cytomegalovirus promoter. It was identified that PDA cells transfected with this plasmid were able to secrete Sema3D at a reduced amount in the absence of ANXA2 (FIG. 20), although the exact mechanism for the secretion of this exogenously overexpressed Sema3D remains to be explored. Panc02 cells transfected with scramble siRNA and/or an empty plasmid were used as a control. ANXA2-targeting siRNA significantly suppressed the invasion of Panc02 cells (FIG. 6A). However, overexpression of Sema3D showed a trend but did not significantly restore the ANXA2 siRNA-suppressed invasion capacity of Panc02 cells. Similarly, the addition of exogenous Sema3D-AP to the culture medium was also able to partially restore the ANXA2 siRNA-suppressed invasion capacity of Panc02 cells (FIG. 21); however, when PLXND1 was also knocked down by siRNA, Sema3D-AP was no longer able to restore this suppressed invasion capacity. This result further suggests that PlxnD1 mediates the role of Sema3D in PDA invasion.

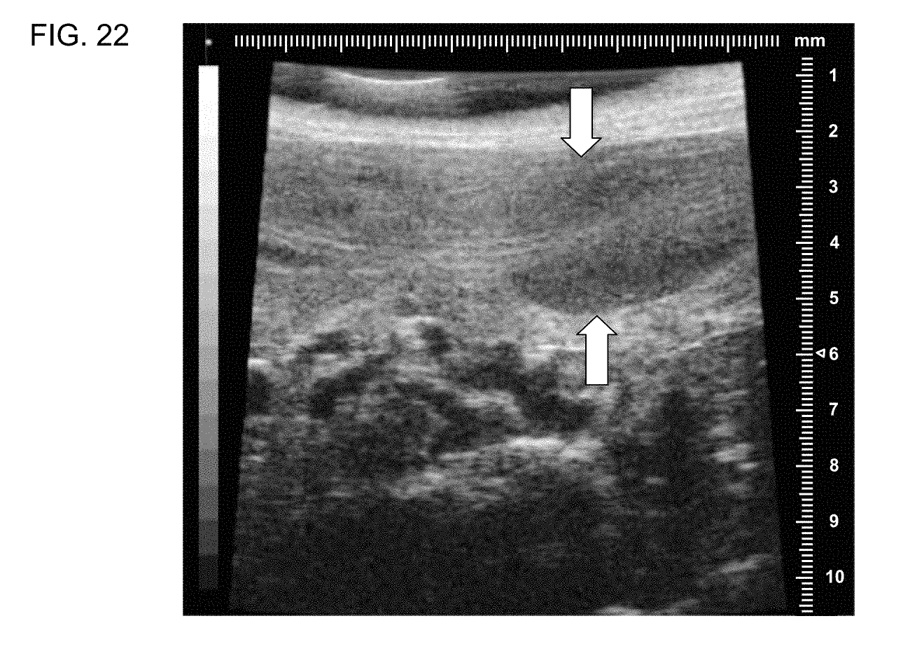

Next, KPCA^{-/-} cells were infected with GFP-encoding lentivirus carrying the mouse Sema3D, and their capacity to form liver metastases was tested in the hemi-spleen model. None of the mice receiving KPCA^{-/-} cells infected with the same lentivirus expressing GFP alone formed liver metastases (FIG. 2). Sema3D overexpression did not alter tumor cell proliferation rate (FIG. 15C) or primary tumor growth (FIG. 15D). However, 11 of 12 mice receiving KPCA^{-/-} cells infected with lentivirus carrying both Sema3D and GFP cDNAs developed liver metastases, assessed mid-assay by ultrasound (FIG. 22) and assessed terminally at necropsy (FIG. 6B), suggesting that reintroduction of Sema3D can largely restore the loss of metastatic potential in KPCA−/− cells. Together, these results suggest that Sema3D and PlxnD1 represent an AnxA2-downstream pathway that mediates the role of AnxA2 in PDA invasion and metastasis formation.

### REFERENCES CITED

The following references are referred to and incorporated by reference above by a corresponding reference number 1 through 42 as listed below set forth within a parentheses ( ) above. The references numbers listed above are not in bold text.
1. R. Siegel, J. Ma, Z. Zou, A. Jemal, Cancer statistics, 2014. CA Cancer J. Clin. 64, 9-29 (2014).
2. D. Li, K. Xie, R. Wolff, J. L. Abbruzzese, Pancreatic cancer. Lancet 363, 1049-1057 (2004).
3. E. Lutz, C. J. Yeo, K. D. Lillemoe, B. Biedrzycki, B. Kobrin, J. Herman, E. Sugar, S. Piantadosi, J. L. Cameron, S. Solt, B. Onners, I. Tartakovsky, M. Choi, R. Sharma, P. B. Illei, R. H. Hruban, R. A. Abrams, D. Le, E. Jaffee, D. Laheru, A lethally irradiated allogeneic granulocyte-macrophage colony stimulating factor-secreting tumor vaccine for pancreatic adenocarcinoma. A phase 11 trial of safety, efficacy, and immune activation. Ann. Surg. 253, 328-335 (2011).
4. L. Zheng, K. Foley, L. Huang, A. Leubner, G. Mo, K. Olino, B. H. Edil, M. Mizuma, R. Sharma, D. T. Le, R. A. Anders, P. B. Illei, J. E. Van Eyk, A. Maitra, D. Laheru, E. M. Jaffee, Tyrosine 23 phosphorylation-dependent cell-surface localization of annexin A2 is required for invasion and metastases of pancreatic cancer. PLOS One 6, e19390 (2011).
5. A. V. Biankin, N. Waddell, K. S. Kassahn, M. C. Gingras, L. B. Muthuswamy, A. L. Johns, D. K. Miller, P. J. Wilson, A. M. Patch, J. Wu, D. K. Chang, M. J. Cowley, B. B. Gardiner, S. Song, I. Harliwong, S. Idrisoglu, C. Nourse, E. Nourbakhsh, S. Manning, S. Wani, M. Gongora, M. Pajic, C. J. Scarlett, A. J. Gill, A. V. Pinho, I. Rooman, M. Anderson, O. Holmes, C. Leonard, D. Taylor, S. Wood, Q. Xu, K. Nones, J. L. Fink, A. Christ, T. Bruxner, N. Cloonan, G. Kolle, F. Newell, M. Pinese, R. S. Mead, J. L. Humphris, W. Kaplan, M. D. Jones, E. K. Colvin, A. M. Nagrial, E. S. Humphrey, A. Chou, V. T. Chin, L. A. Chantrill, A. Mawson, J. S. Samra, J. G. Kench, J. A. Lovell, R. J. Daly, N. D. Merrett, C. Toon, K. Epari, N. Q. Nguyen, A. Barbour, N. Zeps, I. Australian Pancreatic Cancer Genome, N. Kakkar, F. Zhao, Y. Q. Wu, M. Wang, D. M. Muzny, W. E. Fisher, F. C. Brunicardi, S. E. Hodges, J. G. Reid, J. Drummond, K. Chang, Y. Han, L. R. Lewis, H. Dinh, C. J. Buhay, T. Beck, L. Timms, M. Sam, K. Begley, A. Brown, D. Pai, A. Panchal, N. Buchner, R. De Borja, R. E. Denroche, C. K. Yung, S. Serra, N. Onetto, D. Mukhopadhyay, M. S. Tsao, P. A. Shaw, G. M. Petersen, S. Gallinger, R. H. Hruban, A. Maitra, C. A. Iacobuzio-Donahue, R. D. Schulick, C. L. Wolfgang, R. A. Morgan, R. T. Lawlor, P. Capelli, V. Corbo, M. Scardoni, G. Tortora, M. A. Tempero, K. M. Mann, N. A. Jenkins, P. A. Perez-Mancera, D. J. Adams, D. A. Largaespada, L. F. Wessels, A. G. Rust, L. D. Stein, D. A. Tuveson, N. G. Copeland, E. A. Musgrove, A. Scarpa, J. R. Eshleman, T. J. Hudson, R. L. Sutherland, D. A. Wheeler, J. V. Pearson, J. D. McPherson, R. A. Gibbs, S. M. Grimmond, Pancreatic cancer genomes reveal aberrations in axon guidance pathway genes. Nature 491, 399-405 (2012).
6. S. Jones, X. Zhang, D. W. Parsons, J. C. Lin, R. J. Leary, P. Angenendt, P. Mankoo, H. Carter, H. Kamiyama, A. Jimeno, S. M. Hong, B. Fu, M. T. Lin, E. S. Calhoun, M. Kamiyama, K. Walter, T. Nikolskaya, Y. Nikolsky, J. Hartigan, D. R. Smith, M. Hidalgo, S. D. Leach, A. P. Klein, E. M. Jaffee, M. Goggins, A. Maitra, C. Iacobuzio-Donahue, J. R. Eshleman, S. E. Kern, R. H. Hruban, R. Karchin, N. Papadopoulos, G. Parmigiani, B. Vogelstein, V. E. Velculescu, K. W. Kinzler, Core signaling pathways in human pancreatic cancers revealed by global genomic analyses. Science 321, 1801-1806 (2008).
7. M. W. MUller, N. A. Giese, J. M. Swiercz, G. O. Ceyhan, I. Esposito, U. Ilinz, P. Bichlcr, T. Giese, M. W. Blchler, S. Offermanns, H. Friess, Association of axon guidance factor Semaphorin 3A with poor outcome in pancreatic cancer. Int. J. Cancer 121, 2421-2433 (2007).
8. I. Roodink, G. Kats, L. van Kempen, M. Grunberg, C. Maass, K. Verrijp, J. Raats, W. Leenders, Semaphorin 3E expression correlates inversely with plexin D1 during tumor progression. Am. J. Pathol. 173, 1873-1881 (2008).
9. M. A. Shalaby, L. Hampson, A. Oliver, I. Hampson, Plexin D1: New potential biomarker for cervical cancer. J. Immunoassay Immunochem. 33, 223-233 (2012).
10. A. Casazza, V. Finisguerra, L. Capparuccia, A. Camperi, J. M. Swiercz, S. Rizzolio, C. Rolny, C. Christensen, A. Bertotti, I. Sarotto, M. Risio, L. Trusolino, J. Weitz, M. Schneider, M. Mazzone, P. M. Comoglio, L. Tamagnone, Sema3E-Plexin D1 signaling drives human cancer cell invasiveness and metastatic spreading in mice. J. Clin. Invest. 120, 2684-2698 (2010).
11. J. Luchino, M. Hocine, M. C. Amoureux, B. Gibert, A. Bemet, A. Royet, I. Treilleux, P. Lécine, J. P. Borg, P. Mehlen, S. Chauvet, F. Mann, Semaphorin 3E suppresses tumor cell death triggered by the plexin D dependence receptor in metastatic breast cancers. Cancer Cell 24, 673-685 (2013).
12. P. Mehlen, C. Delloye-Bourgeois, A. Chddotal, Novel roles for Slits and netrins: Axon guidance cues as anticancer targets? Nat. Rev. Cancer 11, 188-197 (2011).
13. T. Worzfeld, S. Offermanns, Semaphorins and plexins as therapeutic targets. Nat. Rev. Drug Discov. 13, 603-621 (2014).
14. M. Rehman, L. Tamagnone, Semaphorins in cancer: Biological mechanisms and therapeutic approaches. Semin. Cell Dev. Biol. 24, 179-189 (2013).
15. C. H. Tseng, K. D. Murray, M. F. Jou, S. M. Hsu, H. J. Cheng, P. H. Huang, Sema3E/plexin-D 1 mediated epithelial-to-mesenchymal transition in ovarian endometrioid cancer. PLOS One 6, e19396 (2011).
16. S. R. Hingorani, L. Wang, A. S. Multani, C. Combs, T. B. Deramaudt, R. H. Hruban, A. K. Rustgi, S. Chang, D. A. Tuveson, Trp53R172H and KrasG12D cooperate to promote chromosomal instability and widely metastatic pancreatic ductal adenocarcinoma in mice. Cancer Cell 7, 469-483 (2005).
17. Q. Ling, A. T. Jacovina, A. Deora, M. Febbraio, R. Simantov, R. L. Silverstein, B. Hempstead, W. H. Mark, K. A. Hajjar, Annexin II regulates fibrin homeostasis and neoangiogenesis in vivo. J. Clin. Invest. 113, 38-48 (2004).
18. B. Huang, A. B. Deora, K. L. He, K. Chen, G. Sui, A. T. Jacovina, D. Almeida, P. Hong, P. Burgman, K. A. Hajjar, Hypoxia-inducible factor-1 drives annexin A2 systemmediated perivascular fibrin clearance in oxygen-induced retinopathy in mice. Blood 118, 2918-2929 (2011).
19. K. C. Soares, K. Foley, K. Olino, A. Leubner, S. C. Mayo, A. Jain, E. Jaffee, R. D. Schulick, K. Yoshimura, B. Edil, L. Zheng, A preclinical murine model of hepatic metastases. J. Vis. Exp. 91, 51677 (2014).
20. L. Tamagnone, P. M. Comoglio, To move or not to move? Semaphorin signalling in cell migration. EMBO Rep. 5, 356-361 (2004).
21. V. Gerke, S. E. Moss, Annexins: From structure to function. Physiol. Rev. 82, 331-371 (2002).
22. Y. Biener, R. Feinstein, M. Mayak, Y. Kaburagi, T. Kadowaki, Y. Zick, Annexin II is a novel player in insulin signal transduction. Possible association between annexin II phosphorylation and insulin receptor internalization. J. Biol. Chem. 271, 29489-29496 (1996).
23. D. S. Drust, C. E. Creutz, Aggregation of chromaffin granules by calpactin at micromolar levels of calcium. Nature 331, 88-91 (1988).
24. M. J. Geisow, J. H. Walker, C. Boustead, W. Taylor, Annexins—New family of Ca2+-regulated-phospholipid binding protein. Biosci. Rep. 7, 289-298 (1987).
25. C. Gu, Y. Yoshida, J. Livet, D. V. Reimert, F. Mann, J. Merte, C. E. Henderson, T. M. Jessell, A. L. Kolodkin, D. D. Ginty, Semaphorin 3E and plexin-D1 control vascular pattern independently of neuropilins. Science 307, 265-268 (2005).
26. L. Tamagnone, P. M. Comoglio, Signalling by Semaphorin receptors: Cell guidance and beyond. Trends Cell Biol. 10, 377-383 (2000).
27. Y. S. Oh, P. Gao, K. W. Lee, I. Ceglia, J. S. Seo, X. Zhang, J. H. Ahn, B. T. Chait, D. J. Patel, Y. Kim, P. Greengard, SMARCA3, a chromatin-remodeling factor, is required for p11-dependent antidepressant action. Cell 152, 831-843 (2013).
28. A. J. Minn, G. P. Gupta, P. M. Siegel, P. D. Bos, W. Shu, D. D. Giri, A. Viale, A. B. Olshen, W. L. Gerald, J. Massagué, Genes that mediate breast cancer metastasis to lung. Nature 436, 518-524 (2005).
29. D. I. Rodenhiser, J. D. Andrews, T. A. Vandenberg, A. F. Chambers, Gene signatures of breast cancer progression and metastasis. Breast Cancer Res. 13, 201 (2011).
30. K. Zylbersztejn, M. Petkovic, A. Burgo, M. Deck, S. Garel, S. Marcos, E. Bloch-Gallego, F. Nothias, G. Serini, D. Bagnard, T. Binz, T. Galli, The vesicular SNARE Synaptobrevin is required for Semaphorin 3A axonal repulsion. J. Cell Biol. 196, 37-46 (2012).
31. C. J. Merrifield, U. Rescher, W. Almers, J. Proust, V. Gerke, A. S. Sechi, S. E. Moss, Annexin 2 has an essential role in actin-based macropinocytic rocketing. Curr. Biol. 11, 1136-1141 (2001).
32. U. Yazdani, J. R. Terman, The Semaphorins. Genome Biol. 7, 211 (2006).
33. E. Pecho-Vrieseling, M. Sigrist, Y. Yoshida, T. M. Jessell, S. Arber, Specificity of sensorymotor connections encoded by Sema3e-Plxnd1 recognition. Nature 459, 842-846 (2009).
34. A. A. Bapat, G. Hostetter, D. D. Von Hoff, H. Han, Perineural invasion and associated pain in pancreatic cancer. Nat. Rev. Cancer 11, 695-707 (2011).
35. H. Takahashi, H. Ohigashi, O. Ishikawa, K. Gotoh, T. Yamada, S. Nagata, Y. Tomita, H. Eguchi, Y. Doki, M. Yano, Perineural invasion and lymph node involvement as indicators of surgical outcome and pattern of recurrence in the setting of preoperative gemcitabine-based chemoradiation therapy for resectable pancreatic cancer. Ann. Surg. 255, 95-102 (2012).
36. K. M. Bever, E. A. Sugar, E. Bigelow, R. Sharma, D. Laheru, C. L. Wolfgang, E. M. Jaffee, A. Anders, A. De Jesus-Acosta, L. Zheng, The prognostic value of stroma in pancreatic cancer in patients receiving adjuvant therapy. HPB 17, 292-298 (2015).
37. C. A. Iacobuzio-Donahue, B. Fu, S. Yachida, M. Luo, H. Abe, C. M. Henderson, F. Vilardell, Z. Wang, J. W. Keller, P. Banerjee, J. M. Herman, J. L. Cameron, C. J. Yeo, M. K. Halushka, J. R. Eshleman, M. Raben, A. P. Klein, R. H. Hruban, M. Hidalgo, D. Laheru, DPC4 gene status of the primary carcinoma correlates with patterns of failure in patients with pancreatic cancer. J. Clin. Oncol. 27, 1806-1813 (2009).
38. P. A. Pérez-Mancera, A. G. Rust, L. van der Weyden, G. Kristiansen, A. Li, A. L. Sarver, K. A. Silverstein, R. GrUtzmann, D. Aust, P. Rommele, T. Knösel, C. Herd, D. L. Stemple, R. Kettleborough, J. A. Brosnan, A. Li, R. Morgan, S. Knight, J. Yu, S. Stegeman, L. S. Collier, J. J. ten Hoeve, J. de Ridder, A. P. Klein, M. Goggins, R. H. Hruban, D. K. Chang, A. V. Biankin, S. M. Grimmond, Australian Pancreatic Cancer Genome; L. F. Wessels, S. A. Wood, C. A. Iacobuzio-Donahue, C. Pilarsky, D. A. Largaespada, D. J. Adams, D. A. Tuveson, The deubiquitinase USP9X suppresses pancreatic ductal adenocarcinoma. Nature 486, 266-270 (2012).
39. A. Jain, J. E. Slansky, L. C. Matey, H. E. Allen, D. M. Pardoll, R. D. Schulick, Synergistic effect of a granulocyte-macrophage colony-stimulating factor-transduced tumor vaccine and systemic interleukin-2 in the treatment of murine colorectal cancer hepatic metastases. Ann. Surg. Oncol. 10, 810-820 (2003).
40. T. H. Corbett, B. J. Roberts, W. R. Leopold, J. C. Peckham, L. J. Wilkoff, D. P. Griswold Jr., F. M. Schabel Jr., Induction and chemotherapeutic response of two transplantable ductal adenocarcinomas of the pancreas in C57BL/6 mice. Cancer Res. 44, 717-726 (1984).
41. X. Zhou, Y. Cui, X. Huang, Z. Yu, A. M. Thomas, Z. Ye, D. M. Pardoll, E. M. Jaffee, L. Cheng, Lentivirus-mediated gene transfer and expression in established human tumor antigen-specific cytotoxic T cells and primary unstimulated T cells. Hum. Gene Ther. 14, 1089-1105 (2003).
42. J. Ruan, M. Luo, C. Wang, L. Fan, S. N. Yang, M. Cardenas, H. Geng, J. P. Leonard, A. Melnick, L. Cerchietti, K. A. Hajjar, Imatinib disrupts lymphoma angiogenesis by targeting vascular pericytes. Blood 121, 5192-5202 (2013).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of reducing or inhibiting tumor invasion or tumor metastatic progression in a subject comprising:
identifying a subject having or at risk of developing cancer; and
administering to the subject an effective amount of an agent that reduces the transcription or activity of Semaphorin,
thereby reducing or inhibiting tumor invasion or tumor metastatic progression in said subject.

2. The method of claim 1, wherein said Semaphorin comprises Semaphorin 3D (Sema3D).

3. The method of claim 2, wherein said agent that reduces the transcription or activity of Sema3D comprises a small molecule inhibitor, an antibody or a fragment thereof, or a nucleic acid molecule.

4. The method of claim 3, wherein nucleic acid molecule comprises double stranded ribonucleic acid (dsRNA), small hairpin RNA or short hairpin RNA (shRNA), or antisense RNA, or any portion thereof.

5. The method of claim 1, wherein said agent that reduces the transcription or activity of Sema3D is administered at a dose of 1 mg/kg/day-1 g/kg/day.

6. The method of claim 1, further comprising administering an agent that reduces the transcription or activity of plexin to said subject.

7. The method of claim 4, wherein said plexin comprises plexin D1 (PlxnD1).

8. The method of claim 5, wherein said PlxnD1 inhibitor comprises an anti-PlxnD1 antibody.

9. The method of claim 1, further comprising administering an agent that reduces the transcription or activity of ANXA2 to said subject.

10. The method of claim 7, wherein the ANXA2 inhibitor comprises an anti-ANXA2 antibody.

11. The method of claim 1, wherein the subject has had the bulk of the tumor resected.

12. The method of claim 1, wherein said cancer comprises a gastrointestinal cancer.

13. The method of claim 10, wherein said cancer comprises pancreatic cancer.

14. The method of claim 11, wherein said pancreatic cancer comprises a pancreatic ductal adenocarcinoma (PDA).

15. The method of claim 1, wherein said subject is a human.

16. The method of claim 1, wherein said agent that reduces the transcription or activity of Sema3D and said agent that reduces the transcription or activity of PlxnD1 are administered simultaneously.

17. The method of claim 1, wherein said agent that reduces the transcription or activity of Sema3D and said agent that reduces the transcription or activity of PlxnD1 are administered sequentially.

18. The method of claim 19, wherein said agent that reduces the transcription or activity of Sema3D and said agent that reduces the transcription or activity of PlxnD1 are administered twice per week.

19. The method of claim 1, further comprising administering an anti-cancer agent to said subject.

20. The method of claim 1, wherein said agent that reduces the transcription or activity of Sema3D is administered orally, intravenously, intramuscularly, systemically, subcutaneously or by inhalation.

21. The method of claim 1, wherein the composition is administered in a form selected from the group consisting of pills, capsules, tablets, granules, powders, salts, crystals, liquids, serums, syrups, suspensions, gels, creams, pastes, films, patches, and vapors.

22. A method of screening for a candidate compound which inhibits tumor invasion and/or tumor metastasis, comprising:
contacting a candidate compound with a pancreatic cancer cell;
determining a Sema3D secretion level; and
identifying the candidate compound as a candidate compound for inhibiting tumor invasion and/or tumor metastasis if the candidate compound inhibits secretion of Sema3D.

23. A method of determining whether pancreatic cancer will metastasize in a subject comprising
obtaining a pancreatic tumor sample from a subject;
determining a level of Sema3D in said tumor sample;
comparing said level of Sema3D in said tumor sample to a control level of Sema3D, wherein an increased level of Sema3D in said tumor sample relative to the control level of Sema3D indicates said pancreatic cancer will metastasize in said subject.

24. The method of claim 23, further comprising administering to the subject an effective amount of an agent that reduces the transcription or activity of Sema3D, thereby reducing or inhibiting tumor invasion or tumor metastatic progression in said subject.
